(19) United States
(12) Patent Application Publication
(10) Pub. No.: US 2026/0216062 A1
(43) Pub. Date: Jul. 30, 2026

(54) METHODS AND DEVICES FOR HORMONE REPLACEMENT THERAPY

(71) Applicant: Daré Bioscience, Inc., San Diego, CA (US)

(72) Inventors: David Friend, San Diego, CA (US); Christine Mauck, Arden, NC (US); Nadene Zack, San Diego, CA (US)

(21) Appl. No.: 19/146,096

(22) PCT Filed: Jan. 8, 2024
(86) PCT No.: PCT/US2024/010729

§ 371 Date: Jul. 7, 2025

Related U.S. Application Data
(60) Provisional application No. 63/437,940, filed on Jan. 9, 2023.
(60) Provisional application No. 63/531,469, filed on Aug. 8, 2023.

(51) Int. Cl. A61K 9/00; A61K 31/565; A61K 31/57; A61K 47/32; A61P 15/12

(52) U.S. Cl. CPC A61K 9/0036; A61K 31/565; A61K 31/57; A61K 47/32; A61P 15/12

(57) ABSTRACT

The present invention is in the field of intravaginal rings, in particular segmented, ethylene-vinyl acetate (EVA) intravaginal rings, and their use treating, ameliorating, or preventing, vasomotor symptoms in perimenopause and/or menopausal women and for treating, ameliorating, or preventing vulvar and vaginal atrophy (VVA).

### RELATED APPLICATIONS

This application claims the benefit of priority under 35 U.S.C. § 119 (e) to U.S. Provisional Application No. 63/437,940, filed Jan. 9, 2023 and U.S. Provisional Application No. 63/531,469, filed Aug. 8, 2023, the entire contents of each of which is incorporated herein by reference in their entireties.

### FIELD OF THE INVENTION

The present invention is in the field of intravaginal rings, in particular segmented, ethylene-vinyl acetate (EVA) intravaginal rings, and their use treating, ameliorating, or preventing, vasomotor symptoms in perimenopause and/or menopausal women and for treating, ameliorating, or preventing vulvar and vaginal atrophy (VVA).

### BACKGROUND

Menopause is often associated with vasomotor symptoms (VMS), including hot flushes and night sweats, and these are the most frequently observed symptoms during menopause, although these are not experienced by all women. Additional symptoms may include vaginal symptoms such as dryness and dyspareunia, sleep disturbances, and arthralgia. Menopause is associated with pathophysiology of genitourinary symptoms which is caused by a reduction in estrogen levels. Lower levels of estrogen cause a thinning of the vaginal epithelium, the elasticity of the vagina is reduced, and there is an increase in connective tissue with eventual fibrotic change in some women. Decreased estrogen levels are also associated with a reduction in vaginal blood flow and in vaginal lubrication. These physiological changes lead to vulvovaginal atrophy (VVA) and are responsible for the range of symptoms observed in many menopausal women, including vaginal dryness, vaginal and/or vulvar irritation/itching, dysuria, vaginal pain associated with sexual activity, and vaginal bleeding associated with sexual activity.

Hormone therapy is accepted as an effective treatment for the management of both VMS and symptomatic VVA, with both local and systemic treatments widely used. The use of estrogen for the treatment of symptoms of menopause is advocated by professional medical organizations.

Although estrogen is the most effective treatment for VMS, unopposed treatment (estrogen alone without progesterone) is associated with an increased risk of endometrial hyperplasia and carcinoma in women with an intact uterus, with a meta-analysis of 30 studies showing a relative risk of 2.3 (95% CI of 2.1-2.5) among women using estrogen and those not using it. This risk is reduced by the addition of progestogens, with the incidence of endometrial cancer under combined treatment being no different from that in untreated women. Furthermore, a Cochrane Review noted a greater effect on reducing hot flash severity following treatment with estrogen and progestogens than with estrogen alone.

### SUMMARY

The instant technology generally relates to a method for treating one or more symptoms of menopause in a subject, comprising intravaginally inserting an implantable drug delivery device to the subject, wherein the implantable drug delivery device contains (and releases) an estrogen and a progestin. In embodiments, the implantable drug delivery device provides a baseline-adjusted steady state plasma estrogen concentration between about 15 pg/mL and about 50 pg/mL in the subject.

In embodiments, the subject is a female subject with a uterus.

In embodiments, the one or more symptoms of menopause include vasomotor symptoms (VMS). In embodiments, the one or more symptoms of menopause include genitourinary syndrome. In embodiments, the one or more symptoms of menopause include one or more of: hot flashes, night sweats, vaginal pH, changes in vaginal cytology, vaginal dryness, and vaginal pain. In embodiments, the one or more symptoms of menopause are improved by at least 20%.

In embodiments, the estrogen is estradiol. In embodiments, the estrogen is an estradiol equivalent. In embodiments, the estradiol is bio-identical estradiol.

In embodiments, the progestin is progesterone. In embodiments, the estrogen is a progesterone equivalent. In embodiments, the progesterone is bio-identical progesterone.

In embodiments, the implantable drug delivery device delivers the estrogen and the progestin continuously over a period of time. In embodiments, the period of time is between 7 days and 35 days. In embodiments, the period of time is about 28 days.

In embodiments, the implantable drug delivery device comprises a solid ethylene vinyl acetate polymer matrix.

In embodiments, the implantable drug delivery device comprises an ethylene-vinyl-acetate (EVA), intravaginal ring (IVR), wherein the ring contains at least two segments/fibers, wherein one segment contains the estrogen, and the second segment contains the progestin.

In embodiments, the implantable drug delivery device comprises an inner core comprising one or more active pharmaceutical ingredients distributed throughout a first water-insoluble polymer; an intermediate coating positioned around the inner core, said intermediate coating comprising an acrylate polymer; and an outer coating positioned around the intermediate coating, said outer coating comprising a second water-insoluble polymer.

In embodiments, the acrylate polymer is formed from one or more monomers of formula (I):

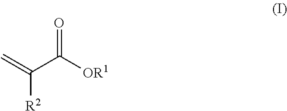

wherein R1 is selected from H, alkyl, alkenyl, alkynyl, or aryl; and R2 is selected from H or alkyl.

In embodiments, the implantable drug delivery device releases the estrogen at a rate of about 50 μg/day to about 200 μg/day. In embodiments, the implantable drug delivery device releases the progestin at a rate of about 1 mg/day to about 15 mg/day. In embodiments, the implantable drug delivery device releases the estrogen at a rate of about 80 μg/day and the progestin at a rate of about 4 mg/day. In embodiments, the implantable drug delivery device releases the estrogen at a rate of about 160 μg/day and the progestin at a rate of about 8 mg/day.

In embodiments, the implantable drug delivery device is removed about 20 days to about 35 days after insertion. In embodiments, a second implantable drug delivery device is inserted after removal of the implantable drug delivery device. In embodiments, the implantable drug delivery device is replaced every 20 to 35 days. In embodiments, the implantable drug delivery device is replaced approximately every 28 days.

In embodiments, the implantable drug delivery device provides a baseline-adjusted steady state plasma estrogen concentration between about 20 pg/mL and about 30 pg/mL in the subject. In embodiments, the implantable drug delivery device provides a baseline-adjusted steady state plasma progesterone concentration between about 1 ng/ml and about 5 ng/mL in the subject. In embodiments, the implantable drug delivery device provides a baseline-adjusted steady state plasma progesterone concentration between about 2 ng/ml and about 4 ng/ml in the subject.

### BRIEF DESCRIPTION OF THE DRAWINGS

FIG. 1. Plasma concentration (base-line adjusted) of E2 following vaginal administration of either the 80/4 or the 160/8 IVR. Data are means±SD (n=10 [80/4 IVR] or 11 [160/8 IVR]).

FIG. 2. Plasma concentration (base-line adjusted) of E1 following vaginal administration of either the 80/4 or the 160/8 IVR. Data are means±SD (n=10 [80/4 IVR] or 11 [160/8 IVR]).

FIG. 3. Plasma concentration (base-line adjusted) of P4 following vaginal administration of either the 80/4 or the 160/8 IVR. Data are means±SD (n=10 [80/4 IVR] or 11 [160/8 IVR]).

FIG. 4. Plasma concentration (base-line adjusted) of E2 (top panel) and E1 (bottom panel) following oral administration of Estrofem (1 mg E2) and Prometrium (100 mg P4). Data are means±SD (n=11).

FIG. 5. Plasma concentration (base-line adjusted) of P4 following oral administration of Estrofem (1 mg E2) and Prometrium (100 mg P4). Data are means±SD (n=11).

FIG. 6. Disposition of Participants.

FIG. 7. Plasma P4 (ng/mL) on indicated days of treatment for 80/4 IVR. Red: cycle 1; Green: cycle 2; Blue: cycle 3; EOT: end of treatment.

FIG. 8. Plasma P4 (ng/mL) on indicated days of treatment for 160/8 IVR.

FIG. 9. Plasma E2 (pg/mL) on indicated days of treatment for 80/4 IVR.

FIG. 10. Plasma E2 (pg/mL) on indicated days of treatment for 160/8 IVR.

FIG. 11. Plasma E1 (pg/mL) on indicated days of treatment for 80/4 IVR.

FIG. 12. Plasma E1 (pg/mL) on indicated days of treatment for 160/8 IVR.

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

The term “estradiol equivalent” includes, without limitation, conjugated estrogen (e.g., Premarin, Cenestin), 17β-estradiol (e.g., Estrace), Estropipate (e.g., Ogen, Ortho-Est), esterified estrogen (e.g., Menest, Estratab), Tri-est/Bi-est, or ethinyl estradiol, estradiol acetate (e.g., Femtrace).

The term “progesterone equivalent” includes, without limitation, Medroxyprogesterone acetate (e.g., Provera), Micronized progesterone (e.g., Prometrium), Norethindron acetate (e.g., Aygestin), and Norethindrone (e.g., Micronor).

### Devices

Disclosed herein are IVRs that allow for estrogen (e.g., E2) and progestin (e.g., P) to be integrated into a single ethylene-vinyl acetate (EVA) ring-delivery system. The IVRs disclosed herein can be used to treat (e.g., cure, suppress), ameliorate, and/or prevent (e.g., delaying or preventing die onset, recurrence or relapse of) one or more symptoms of perimenopause or menopause, such as without limitation VMS in women with an intact uterus, while also aiming to treat, ameliorate, an/or prevent the symptoms of VVA. VMS symptoms can include, without limitation, one or more of the following: hot flashes, flushing, night sweating, mood swings, anxiety, urinary incontinence, cognitive disturbances (memory loss, problems with concentration, arthralgia, weight gain, sexual dysfunction, vaginal disturbances, sleep disturbances, such as insomnia, bone loss, heart disease, atherosclerosis, and heart palpitations. VVA symptoms can include, without limitation, one or more of dryness, burning, itching, vaginal discomfort, vaginal discharge, pain and burning when urinating, urgency with urination, increased urinary tract infections, urinary incontinence, dyspareunia, discomfort with intercourse, decreased vaginal lubrication during sexual activity, shortening and tightening of the vaginal canal, and spotting during intercourse.

The disclosed IVRs are designed to either deliver about 80 to about 160 μg/day E2 with an adequate dose of P for endometrial protection over a 28-day period. Previous reports using IVRs comprised of an inner core of silicone elastomer matrix containing E2 and P, and a silicone elastomer overcoat, have demonstrated the effectiveness of delivering 160 μg/day E2 with either 10 or 20 mg/day P in 20 postmenopausal women. Results showed a significant reduction in the incidence of hot flashes and night sweats from 2 weeks through 16 weeks, with ultrasound monitoring of the endometrium suggesting effective protection against endometrial hyperplasia.

In some embodiments of the invention described herein, where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. For example, a range from 27% to 36% would include 27% to 29%, or 27% to 33% or 33% to 35%, etc. Such ranges would also include individual points in the range, for example, 28%, 29%, 30% etc.

The disclosed IVRs are made by a process described in U.S. Patent Pub. US20210007976, which is incorporated herein by reference in its entirety. The process comprises compounding pellets, extrusion of fibers followed by joining of the fibers by heat welding. Blending can be accomplished using a Turbula mixer (Model T 10 B, with a 17-liter stainless steel mixing vessel, Glenn Mills, Clifton, N.J.). The resulting blends were then compounded by hot-melt extrusion using a twin-screw extruder (Pharma 11 Twin Screw Hot Melt Extruder with a Pharma 11 gravimetric feeder) and fed onto a Pharma 11 Air Cooled conveyor followed by pelletization using a Pharma 11 Vericut Pelletizor (Thermo Fisher Scientific, Dreieich, Germany). The pellets were formed into fibers by hot melt extrusion using a 25 mm single screw extruder (Dr Collin, Ebersberg, Germany). The resulting fibers were cut using a Dr Collin in-line Cutting Station. Cut fibers (or segments) were welded using Automationspartner single station laboratory welder (Ramlosa, Sweden).

IVRs capable of releasing E2 (EP, Aspen Oss B.V., Oss, The Netherlands) at the desired rates were prepared by using fibers of varying length and drug loading. In some embodiments, the IVRs described herein release E2 at rate pf about 160 μg/d and P released at about 4 mg/d (160/4 IVR) or about 8 mg/d (160/8 IVR). In some embodiments, the described IVRs are about 57 mm in overall diameter with a cross sectional diameter of about 5 mm.

The EVA fiber containing E2 was prepared at a concentration of about 10 w/w in EVA (28% vinyl acetate content, Vitaldose®, Celanese Corporation, Boucherville, Canada). The E2 fiber length in the finished IVR is about 15 mm in both the 160/4 and 160/8 IVRs. IVRs releasing about 4 and about 8 mg/d P were prepared using EVA (28% vinyl acetate content, Vitaldose) with a final drug loading of about 27% w/w. To create the 160/4 IVR, the P-containing segment length was about 74.5 mm with a placebo segment length of about 74 mm. To create the 160/8 IVR, 27% loaded EVA fiber was about 148.5 mm in length. There was no placebo segment in the 160/8 IVR.

In an aspect disclosed herein, IVR rings as described herein are used to treat VMS and/or VVA symptoms. In some embodiments of this aspect, the IVR rings contain one or more segments containing E2. In some embodiments, the IVR rings contain one or more segments containing P. In some embodiments, the IVR rings contains both P and E2 each in a separate segment or segments.

In some aspects, the disclosed IVRs are about 57 mm in overall diameter with a cross sectional diameter of about 5 mm.

In some aspects the EVA fiber (segment) containing E2 is prepared at a concentration of about 10 w/w in EVA (28% vinyl acetate content, Vitaldose®, Celanese Corporation, Boucherville, Canada or Polysciences, Inc., Warrington, Pa.) or equivalent from other manufacturers of vinyl acetate. In some embodiments, the E2 fiber length in the finished IVR is about 15 mm.

In some aspects, IVRs releasing about 4 mg and about 8 mg/d P are prepared using EVA (28% vinyl acetate content, Vitaldose) with a final drug loading of about 27% w/w. In some embodiments, the P-containing segment length is about 74.5 mm. In some embodiments, the P-containing segment length is about 148.5 mm in length.

In some embodiments, the disclosed rings can effectively release E2 and/or P for a period of about 7 days or more, or a period of about 10 days or more, or a period of about 14 days or more, or a period of about 20 days or more, or for a period of about 26 days, or for a period of about 27 days, or for a period for about 28 days, or for a period for about 29 days, or for a period of about 30 days, or for a period up to 30 days, or for a period up to 29 days, or for a period up to 28 days.

In embodiments, the rings release E2 and/or P continuously for a period of time. As used herein, the term “continuously” or “continuous release” refers to continuous and uninterrupted (or substantially uninterrupted) release of the drug from the formulation or device and delivering such drug in a continuous manner.

### Methods

The instant technology generally relates to a method for treating one or more symptoms of menopause in a subject, comprising intravaginally inserting an implantable drug delivery device to the subject, wherein the implantable drug delivery device contains (and releases) an estrogen and a progestin.

In embodiments, the implantable drug delivery device provides a baseline-adjusted steady state plasma estrogen concentration between about 15 pg/mL and about 50 pg/mL in the subject. In embodiments, the implantable drug delivery device provides a baseline-adjusted steady state plasma estrogen concentration between about 20 pg/mL and about 30 pg/mL in the subject. In embodiments, the implantable drug delivery device provides a baseline-adjusted steady state plasma estrogen concentration between about 15 pg/mL and about 40 pg/mL in the subject. In embodiments, the implantable drug delivery device provides a baseline-adjusted steady state plasma estrogen concentration between about 15 pg/mL and about 30 pg/mL in the subject. In embodiments, the implantable drug delivery device provides a baseline-adjusted steady state plasma estrogen concentration between about 15 pg/mL and about 20 pg/mL in the subject. In embodiments, the implantable drug delivery device provides a baseline-adjusted steady state plasma estrogen concentration between about 20 pg/mL and about 50 pg/mL in the subject. In embodiments, the implantable drug delivery device provides a baseline-adjusted steady state plasma estrogen concentration between about 20 pg/mL and about 40 pg/mL in the subject

In embodiments, the implantable drug delivery device provides a baseline-adjusted steady state plasma progesterone concentration between about 1 ng/mL and about 5 ng/mL in the subject. In embodiments, the implantable drug delivery device provides a baseline-adjusted steady state plasma progesterone concentration between about 2 ng/ml and about 4 ng/mL in the subject. In embodiments, the implantable drug delivery device provides a baseline-adjusted steady state plasma progesterone concentration between about 1 ng/ml and about 4 ng/ml in the subject. In embodiments, the implantable drug delivery device provides a baseline-adjusted steady state plasma progesterone concentration between about 1 ng/ml and about 3 ng/mL in the subject. In embodiments, the implantable drug delivery device provides a baseline-adjusted steady state plasma progesterone concentration between about 1 ng/ml and about 2 ng/ml in the subject. In embodiments, the implantable drug delivery device provides a baseline-adjusted steady state plasma progesterone concentration between about 2 ng/ml and about 3 ng/mL in the subject. In embodiments, the implantable drug delivery device provides a baseline-adjusted steady state plasma progesterone concentration between about 2 ng/mL and about 5 ng/ml in the subject.

In embodiments, the implantable drug delivery device provides a base-line adjusted steady state plasma estrogen concentration between about 15 pg/mL and about 50 pg/mL in the subject and a baseline-adjusted steady state plasma progesterone concentration between about 2 ng/ml and about 4 ng/ml in the subject. In embodiments, the implantable drug delivery device provides a base-line adjusted steady state plasma estrogen concentration between about 20 pg/mL and about 30 pg/mL in the subject and a baseline-adjusted steady state plasma progesterone concentration between about 1 ng/ml and about 5 ng/ml in the subject.

In embodiments, the subject is a female subject. In embodiments, the subject has a uterus. In embodiments, the subject is post-menopausal. In embodiments, the subject is peri-menopausal.

In embodiments, the one or more symptoms of menopause include vasomotor symptoms (VMS). In embodiments, the one or more symptoms of menopause include genitourinary syndrome. In embodiments, the one or more symptoms of menopause include one or more of: hot flashes, night sweats, vaginal pH, changes in vaginal cytology, vaginal dryness, and vaginal pain. In embodiments, the one or more symptoms of menopause include hot flashes. In embodiments, the one or more symptoms of menopause include night sweats. In embodiments, the one or more symptoms of menopause include abnormal vaginal pH. In embodiments, the one or more symptoms of menopause include changes in vaginal cytology. In embodiments, the one or more symptoms of menopause include vaginal dryness. In embodiments, the one or more symptoms of menopause include vaginal pain.

In embodiments, the one or more symptoms of menopause are improved by at least 20%. In embodiments, the one or more symptoms of menopause are improved by 20% to 100%. In embodiments, the one or more symptoms of menopause are improved by at least 25%. In embodiments, the one or more symptoms of menopause are improved by at least 30%. In embodiments, the one or more symptoms of menopause are improved by at least 40%. In embodiments, the one or more symptoms of menopause are improved by at least 50%. In embodiments, the one or more symptoms of menopause are improved by at least 60%. In embodiments, the one or more symptoms of menopause are improved by at least 70%. In embodiments, the one or more symptoms of menopause are improved by at least 75%. In embodiments, the one or more symptoms of menopause are improved by at least 80%. In embodiments, the one or more symptoms of menopause are improved by at least 90%. In embodiments, the one or more symptoms of menopause are improved by 20% to 90%. In embodiments, the one or more symptoms of menopause are improved by 30% to 90%. In embodiments, the one or more symptoms of menopause are improved by 40% to 90%. In embodiments, the one or more symptoms of menopause are improved by 50% to 90%.

Improvement in one or more symptoms may be measured by any suitable method. For example, The Menopausal Quality of Life Survey (MENQOL) may be used, which surveys not only parameters of VMS, but also physical, psychosocial and sexual symptoms. Other measures include measurement of vaginal pH and cytologic tests of the vaginal epithelium (vaginal maturation index).

In embodiments, the implantable drug delivery device releases the estrogen at a rate of about 50 μg/day to about 200 μg/day. In embodiments, the implantable drug delivery device releases the estrogen at a rate of about 70 μg/day to about 180 μg/day. In embodiments, the implantable drug delivery device releases the estrogen at a rate of about 80 μg/day to about 160 μg/day.

In embodiments, the implantable drug delivery device releases the progestin at a rate of about 1 mg/day to about 15 mg/day. In embodiments, the implantable drug delivery device releases the progestin at a rate of about 2 mg/day to about 10 mg/day. In embodiments, the implantable drug delivery device releases the progestin at a rate of about 4 mg/day to about 8 mg/day.

In embodiments, the implantable drug delivery device releases the estrogen at a rate of about 50 μg/day to about 200 μg/day and the progestin at a rate of about 1 mg/day to about 15 mg/day. In embodiments, the implantable drug delivery device releases the estrogen at a rate of about 70 μg/day to about 180 μg/day and the progestin at a rate of about 2 mg/day to about 10 mg/day. In embodiments, the implantable drug delivery device releases the estrogen at a rate of about 80 μg/day to about 160 μg/day and the progestin at a rate of about 4 mg/day to about 8 mg/day. In embodiments, the implantable drug delivery device releases the estrogen at a rate of about 80 μg/day and the progestin at a rate of about 4 mg/day. In embodiments, the implantable drug delivery device releases the estrogen at a rate of about 160 μg/day and the progestin at a rate of about 8 mg/day.

In embodiments, the implantable drug delivery device is removed about 20 days to about 35 days after insertion. In embodiments, the implantable drug delivery device is removed about 25 days to about 30 days after insertion. In embodiments, the implantable drug delivery device is removed about 25 days after insertion. In embodiments, the implantable drug delivery device is removed about 26 days after insertion. In embodiments, the implantable drug delivery device is removed about 27 days after insertion. In embodiments, the implantable drug delivery device is removed about 28 days after insertion. In embodiments, the implantable drug delivery device is removed about 29 days after insertion. In embodiments, the implantable drug delivery device is removed about 30 days after insertion.

In embodiments, a second implantable drug delivery device is inserted after removal of the implantable drug delivery device.

In embodiments, the implantable drug delivery device is replaced every 20 to 35 days. In embodiments, the implantable drug delivery device is replaced every 25 to 30 days. In embodiments, the implantable drug delivery device is replaced approximately every 25 days. In embodiments, the implantable drug delivery device is replaced approximately every 26 days. In embodiments, the implantable drug delivery device is replaced approximately every 27 days. In embodiments, the implantable drug delivery device is replaced approximately every 28 days. In embodiments, the implantable drug delivery device is replaced approximately every 29 days. In embodiments, the implantable drug delivery device is replaced approximately every 30 days.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

One skilled in the art would understand that descriptions of making and using the intravaginal rings described herein is for the sole purpose of illustration, and that the present disclosure is not limited by this illustration.

### Example 1. Phase 1/2 Clinical Pharmacokinetic and Safety Study: 1 Month

DARE-HRT1 is an ethylene vinyl acetate (EVA) copolymer intravaginal ring (IVR) that releases bioidentical 17ß estradiol and progesterone over 28 days of use and is being developed for the following indications: 1) treatment of moderate-to-severe VMS associated with menopause in women with an intact uterus; and 2) reduction in the incidence of symptomatic VVA in women requiring treatment for VMS due to menopause.

To understand the performance of DARE-HRT1, a Phase 1 clinical study was conducted in Australia. The primary objective of the study was to describe the pharmacokinetic (PK) parameters over 28 days of 2 different dose combinations: 1) 17β-estradiol (E2) at a daily release rate of 80 μg/d and progesterone (P4) at a daily release rate of 4 mg/d (80/4 IVR); and 2) E2 at a daily release rate of 160 μg/d and P4 at 8 mg/day (160/8 IVR). The secondary objectives of the study were to assess the safety and tolerability of each IVR and to compare the systemic exposure of E2, and its metabolite estrone (E1), and P4 to once-daily oral Estrofem®/Prometrium® over 28 days.

### Materials and Methods

The IVRs were manufactured at QPharma (now Sever Pharma Solutions) in Malmö, Sweden. All IVRs were 57 mm in overall diameter and had a cross sectional diameter of 5 mm. They were manufactured as described previously. Briefly, the IVRs were prepared by compounding pellets of EVA (28% vinyl acetate content) with varying concentrations of E2 or P followed by hot-melt extrusion into fibers of various lengths. Fibers were joined by heat welding to create IVRs with the desired release rates. The in vitro release rates of the 80/4 and 160/8 IVRs were described previously. Weiss H, et al. J of Pharma. Sci. 2019; 108 (8): 2677-84.

This was a randomized, open-label, 3-arm, parallel group study in approximately 30 healthy postmenopausal women with intact uteri conducted at two sites in Australia (Keogh Institute for Medical Research, Nedlands, Western Australia, 6009, and PARC Clinical Research, Adelaide, South Australia, 5000). The study was designed to assess the PK of E2, E1, and P4 from DARE-HRT1 IVRs at 2 dose strengths (80/4 or 160/8 IVRs). Oral E2 1 mg (Estrofem®)/P4 100 mg (Prometrium®) once daily for 29 days served as the active reference. The main inclusion criteria admitted healthy postmenopausal female participants (postmenopausal defined as 12 months of spontaneous amenorrhea or 6 months of spontaneous amenorrhea with serum FSH levels>40 mIU/mL, or 6 weeks postsurgical bilateral oophorectomy without hysterectomy (although subjects who have had a hysterectomy are not eligible for this study), with a body mass index of ≥18 and ≤38 kg/m², normal cervix and vagina, intact uterus, current on all Australian screening requirements for cervical cancer, normal mammogram within 24 months of screening, and without known hypersensitivity to E2 or P4, or the components of the IVR.

At the screening visit, the following assessments were performed or information collected: serology (HIV, Hepatitis B and C), follicle stimulating hormone level, drug/alcohol screening, transvaginal ultrasound (TVU), urine dipstick, PK blood sample, and prior and concomitant medications. A similar schedule of visits occurred in the oral arm although there was one less visit compared to the IVR arms.

Plasma sampling occurred on Day 1 pre-dose at −1, −0.5, and 0 hours (immediately; within ±5 minutes) prior to insertion of IVR and at 0.5, 1, 2, 4, 8, 12, 24, and 48 hours following insertion. Subjects were discharged from the clinic after safety assessments had been completed on Day 3. On the morning of Days 8, 11, 15, and 22 the subjects returned to the clinic for a single PK blood draw and safety assessments. Subjects returned to the clinic on Day 28 for a PK blood draw, safety assessments, confirmation of IVR placement, and to begin confinement. On the morning of Day 29 a PK blood sample was collected. The IVR was then removed and PK samples were collected at 0.5, 1, 2, 4, 8, 12, and 24 hours post removal.

Those enrolled in the oral arm (Estrofem/Prometrium) also began dosing on Day 1, concluding with a last dose on Day 29. Doses were administered in the clinic each of the mornings of Day 1 and Days 28 and 29 with a moderate fat breakfast. Plasma sampling occurred on Day 1 pre-dose at −1, −0.5, and 0 (immediately; within ±5 minutes) hours prior to oral dose administration and 0.25, 0.5, 1, 2, 4, 6, 8, 10, 12, and 24 hours post-dose. Subjects were discharged from the clinic and instructed to self-administer the medication in the morning with a meal and approximately 240 mL of water, starting on Day 2 through Day 27, at approximately the same time. On Days 8, 15, and 22, subjects returned to the clinic for a single PK blood draw and safety assessments. Subjects returned on Day 28 and were admitted to consume their oral dose with breakfast and began PK sampling prior to (0 hours) dosing on Day 29 and the following timepoints post oral dose: 0.25, 0.5, 1, 2, 4, 6, 8, 10, 12, and 24 hours.

Plasma samples were analyzed using two validated bioanalytical methods (one for E2 and E1 and one for P4) at Agilex Biolabs Pty Ltd, Thebarton, South Australia, 5031, Australia. The lower limit of quantitation (LLOQ) of E2 was 2.00 pg/mL and 5.00 pg/mL for E1. The LLOQ for P4 was 25 pg/mL.

The IVRs used in this clinical trial were returned to Sever Pharma Solutions following sterilization to determine the residual amount of both E2 and P4 in both IVRs. A validated assay method was used to extract and quantify the drug content in the rings. The remaining levels of drug were compared to the initial quantity of each drug to determine the amount released over 28 days of in vivo use.

After imputation of non-quantifiable concentrations, concentrations were summarized when at least 3 participants within a group had non-missing data at the time point being summarized. Within individual participant concentration versus time profiles, whether graphical or tabular, all assayed concentrations were presented, even when not summarized elsewhere. With respect to PK parameters, summaries were only presented when at least 3 participants had non-missing data within a treatment group.

For each of the treatment arms, plasma E2, E1, and P4 concentrations were summarized for the PK Population (defined as all participants who received a full course of study treatment and who had sufficient concentration data for determination of PK parameters (primary endpoints); data were analyzed according to the actual study treatment received) by descriptive statistics of n, arithmetic mean, SD, CV, median, minimum, and maximum.

The following PK parameters were determined from the plasma concentration-time profiles: time-weighted average observed plasma concentration over 24 hours (Day 29 PK profile for oral dosing only; Cavg), steady-state concentration (IVR dosing only; Css), maximum observed plasma concentration (Cmax), concentration at the end of the dosing interval (tau) for oral dosing only (Ctau), the time that Cmax was observed (tmax), area under the plasma concentration-time curve (AUC) from time 0 to 24 hours for oral dosing only (AUC0-24), AUC from time 0 (Day 1) to the time of the last quantifiable concentration on Day 30 for IVR dosing only (AUCD1-D30), and effective half-life for oral dosing only (t½, eff). These parameters ware summarized descriptively for the PK Evaluable Population (defined as all participants in the PK Population who completed the study and who did not have any major protocol violation(s) and/or treatment compliance issues that may have confounded the interpretation of PK) by treatment group, including n, arithmetic mean, SD, CV, GM, and geometric CV, median, minimum, and maximum. The geometric CV was calculated as the square root of the exponentiated SD of the natural log transformed data (SQRT [exp (sln2)−1]) where appropriate. For tmax, only n, minimum, median, and maximum are reported.

Baseline correction, using the average of the 3 pre-dose concentrations on Day 1, were performed prior to the calculation of PK parameters by noncompartmental methods. Cmax (and tmax) were computed over each 24-hour PK profile (Days 1 and 29) and, for IVR administration, over the entire 28-day treatment interval.

Statistical analysis used the PK Evaluable Population. The Safety Population, defined as all screened participants who received active treatment, i.e., inserted an IVR (and thus were exposed to 1 of the IVRs) or who took at least 1 dose of the oral reference, was used for all listings. The individual sampling and participant concentration-time for plasma concentration of E2, E1, and P4 for the Safety Population was listed and displayed graphically on linear and semi-log scales for each different treatment arm. The drug concentrations were summarized descriptively for the PK Population by treatment group and nominal time point in tabular and graphical formats (linear and semi-log scales).

Assessment and documentation of adverse events (AEs) and concomitant medications occurred at each visit. Safety analyses were conducted on the Safety Population and were performed for all safety variables specified. All AEs were coded by primary system organ class (SOC) and preferred term (PT) according to the MedDRA Version 24.0 or later and presented by participant in data listings. A Treatment Emergent AE (TEAE) was defined as any AE that began or worsened after the first dose of the study drug. A treatment-related AE was defined as any TEAE that was possibly or definitely related to study drug.

### Results

A total of 30 healthy, postmenopausal female participants (n=10 per arm) were planned for inclusion in this study. Overall, 34 female participants were screened and randomized, and 31 participants (91.2%) completed the study. All 34 screened participants (100%) were included in the Screened Population and Randomized Population. Thirty-three participants (97.1%) were included in the Safety Population, and 32 participants (94.1%) were included in the PK and PK Evaluable Populations. Table 1 lists selected demographic as well as some baseline information about the subjects (Safety Population) in the study.

TABLE 1
Summary of Demographic Data and Baseline Characteristics (Safety Population)

| | Treatment group | | | |
|---|---|---|---|---|
| | | | Oral | |
| Parameter | 80/4 IVR | 160/8 IVR | Comparator^{a} | Overall |
| Age | | | | |
| Number (n) | 10 | 12 | 11 | 33 |
| Mean (SD) | 58.8 (6.66) | 56.0 (3.72) | 57.2 (4.42) | 57.2 (4.97) |
| Median (Min, Max) | 58.0 (47, 69) | 55.5 (50, 63) | 57.0 (51, 64) | 57.0 (47, 69) |
| Weight | | | | |
| Number (n) | 10 | 12 | 11 | 33 |
| Mean (SD) | 72.1 (10.0) | 80.1 (15.5) | 77.0 (10.9) | 76.6 (12.6) |
| Median (Min, Max) | 71.6 (58.1, 84.6) | 73.5 (64.4, 106) | 73.5 (65.4, 98.2) | 73.5 (58.1, 106) |
| Height (cm) | | | | |
| Number (n) | 10 | 12 | 11 | 33 |
| Mean (SD) | 160 (3.97) | 165 (5.73) | 165 (3.52) | 163 (5.06) |
| Median (Min, Max) | 160 (151, 164) | 166 (153, 174) | 164 (158, 171) | 164 (1.51, 174) |
| Body Mass | | | | |
| Index (kg/m²) | | | | |
| Number (n) | 10 | 12 | 11 | 33 |
| Mean (SD) | 28.2 (3.20) | 29.3 (4.98) | 28.2 (3.18) | 28.6 (3.86) |
| Median (Min, Max) | 27.3 (24.1, 32.8) | 27.3 (23.5, 37.9) | 27.8 (24.4, 34.0) | 27.5 (23.5, 37.9) |
| Vaginal pH | | | | |
| Number (n) | 10 | 12 | 11 | 33 |
| Mean (SD) | 5.16 (0.75) | 5.32 (0.63) | 5.61 (0.94) | 5.37 (0.78) |
| Median (Min, Max) | 4.85 (4.4, 6.0) | 5.00 (4.4, 6.0) | 5.70 (4.0, 7.0) | 5.30 (4.0, 7.0) |
| Cervical Screening | | | | |
| Test (n [%]) | | | | |
| Normal | 9 (90.0) | 11 (91.7) | 11 (100) | 31 (93.9) |
| Abnormal | 0 | 0 | 0 | 0 |
| Not Collected | 1 (10.0) | 1 (8.3) | 0 | 2 (6.1) |

^{a}Oral Comparator Oral Estrofem/Prometrium QD for 29 days (estradiol 1 mg tablet/progesterone 100 mg capsule)

The residual drug levels in the rings removed on Day 29 are shown in Table 2. Both the 80/4 and 160/8 IVRs released a little over 10% of the total amount of E2 loaded into the rings while the amount of P4 released was about 17% of the total drug loaded into the rings.

TABLE 2
Residual drug levels in the IVRs following removal on Day 29

| | 80/4 IVR | | | | 160/8 IVR | | | |
|---|---|---|---|---|---|---|---|---|
| | E2 (15.7 mg)^{a} | | P4 (395 mg) | | E2 (31.4 mg) | | P4 (787 mg) | |
| | Amount | | Amount | | Amount | | Amount | |
| | (mg) | Assay (%) | (mg) | Assay (%) | (mg) | Assay (%) | (mg) | Assay (%) |
| Mean | 13.9 | 88.5 | 327 | 82.7 | 28.5 | 90.7 | 651 | 82.7 |
| SD | 0.2 | 1.4 | 7.3 | 1.8 | 0.7 | 2.2 | 19.1 | 2.4 |
| % RSD | 1.6 | 1.6 | 2.2 | 2.2 | 2.4 | 2.4 | 2.9 | 2.9 |

^{a}Value in parentheses is the average amount of drug present in the IVR

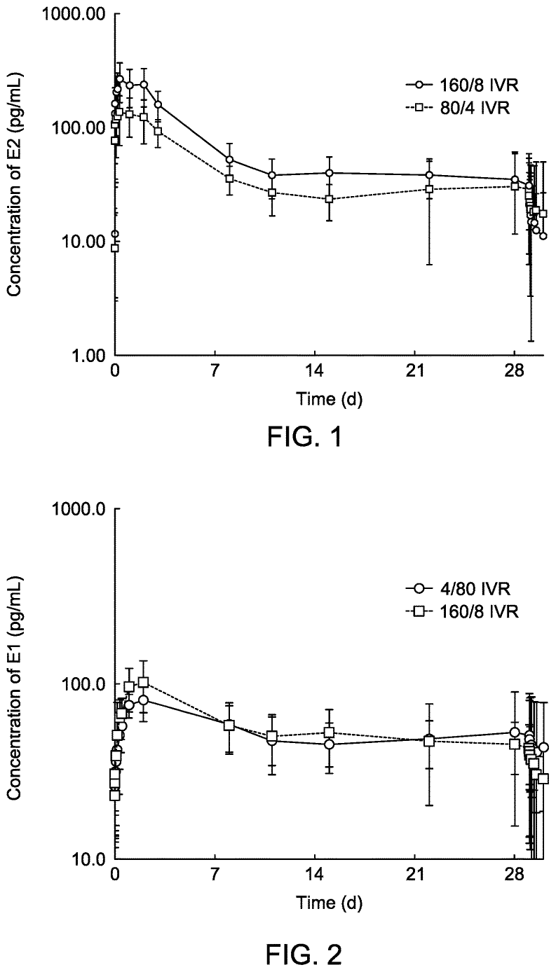

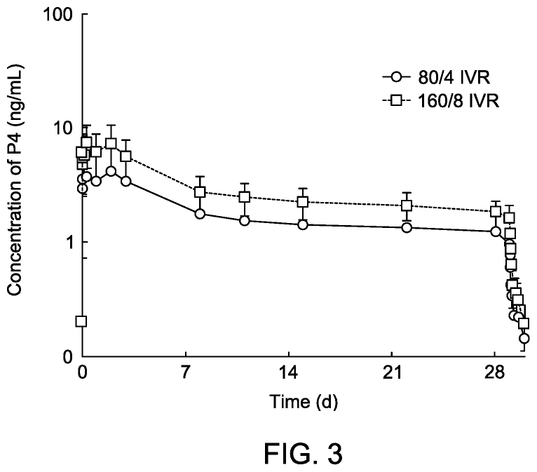

The plasma concentration over time profiles of E2 from the 80/4 IVR and the 160/8 IVR are shown in FIG. 1. Plasma concentrations of E1 from the respective IVRs are shown in FIG. 2. The plasma concentration over time profile for P4 from the two IVRs is shown in FIG. 3. Following the insertion of the IVR on Day 1, plasma concentrations of estradiol, estrone, and progesterone followed a similar pattern: an initial increase in concentrations, peaking within 1 2 days after IVR insertion followed by a decline in concentrations up to approximately Day 8 (192 hours), after which concentrations were maintained until the removal of the IVR on the morning of Day 29. Post-removal of the IVR, concentrations declined towards baseline levels. Concentrations of estradiol, estrone, and progesterone were generally higher for 160/8 IVR compared to 80/4 IVR, consistent with the higher dose of both E2 and P4 in the 160/8 IVR, with the exception of the steady-state concentrations of estrone which were very similar between Day 8 and the morning of Day 29 for both IVRs.

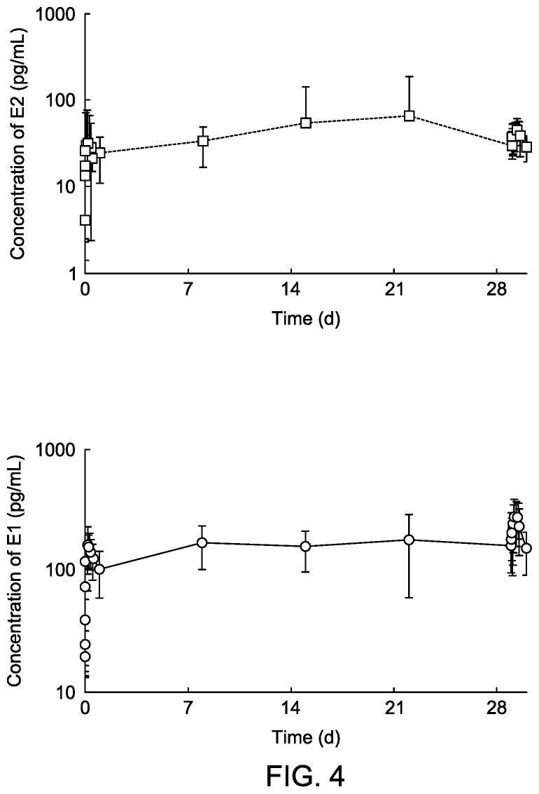

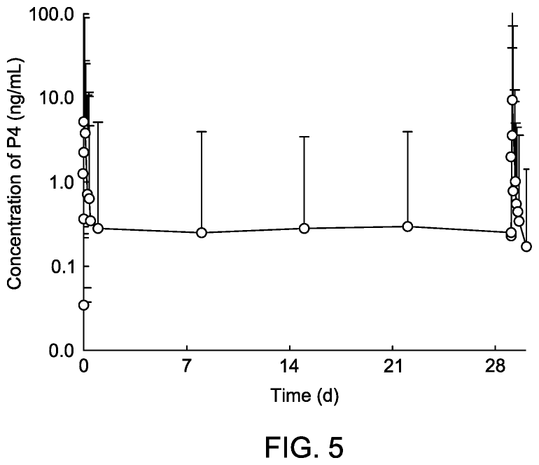

Plasma concentrations of E2, E1, and P4 from the oral arm are shown in FIGS. 4 and 5. For the oral arm, there was the expected increase in estradiol, estrone, and progesterone concentrations on both Day 1 and Day 30, which had returned close to baseline/pre-dose levels within 24 hours. For E2, E1, and P4, steady-state concentrations appeared to have been achieved by Day 8 and were maintained for the duration of the 29 days of dosing. Trough concentrations of E1 were more variable than either E2 or P4 with steady-state appearing to have been achieved between Days 8 and 15.

Various PK parameters for the IVR treatments are shown in Table 3. For all 3 analytes, there was an increase in exposure associated with 160/8 IVR compared to 80/4 IVR for both unadjusted and baseline-adjusted Cmax and AUCD1-D30, although the increase was generally less than the 2-fold increase in the amount of both E2 and P4 in IVR 2 compared to IVR 1. For both E1 and P4, higher Css was also associated with 160/8 IVR but for E2, Css was similar for 80/4 IVR and 160/8 IVR. For both E1 and P4, peak concentrations were observed within 24 hours of insertion, whereas peak E2 concentrations were observed 48 hours after insertion.

For both unadjusted and baseline-corrected estradiol Cmax and Css, there was a less than dose proportional increase, as indicated by the slope estimates (data not shown) being less than 1. For uncorrected and baseline-corrected progesterone Cmax, the slope estimate was closer to 1 and the 90% CI for the estimate contained 1, indicating an approximate dose-proportional increase in exposure after treatment by IVR. There was a less than dose proportional increase in both unadjusted and base line corrected P4 Css.

Various PK parameters for the oral comparator arm are shown in Table 4. There was the expected increase in E2, E1, and P4 concentrations on both Day 1 and Day 30, which had returned close to baseline/pre-dose levels within 24 hours. For both E2 and P4, steady-state concentrations appeared to have been achieved by Day 8 and were maintained for the duration of the 29 days of dosing. Trough concentrations of estrone were more variable than either estradiol or progesterone with steady-state appearing to have been achieved between Days 8 and 15. For both unadjusted and baseline-corrected E2 and E1, both peak and overall exposure were higher on Day 29 compared to Day 1 of oral treatment. Unadjusted and baseline-corrected progesterone exposures were also higher on Day 29 compared to Day 1, but not to the same extent as for either E2 or E1. On both Day 1 and Day 29, peak P4 levels occurred earlier than for either E2 or E1. The estimated effective t½ on Day 29 for both estradiol and estrone was much longer than the corresponding estimate for P4.

TABLE 3
Summary of Plasma PK Parameters by IVR Treatment Arm (Pharmacokinetic Evaluable Population)

| | | IVR Treatment Arm | | | | | |
|---|---|---|---|---|---|---|---|
| | | E2 | | E1 | | P4 | |
| Parameter | | 80/4 IVR | 160/8 IVR | 80/4 IVR | 160/8 IVR | 80/4 IVR | 160/8 IVR |
| (unit) | Statistic | N = 10 | N = 11 | N = 10 | N = 11 | N = 10 | N = 11 |
| Cₘₐₓ (pg/mL | n | 10 | 11 | 10 | 11 | 10 | 11 |
| or ng/mL)^{a} | Mean | 143.655 | 294.154 | 57.213 | 82.961 | 4.527 | 8.984 |
| Baseline- | GM^{b} | 129.244 | 284.552 | 50.929 | 76.241 | 4.010 | 8.402 |
| adjusted Conc. | CV (%)^{c} | 51.51 | 25.41 | 48.86 | 40.05 | 56.51 | 35.81 |
| Cₘₐₓ (pg/mL | n | 10 | 11 | 10 | 11 | 10 | 11 |
| or ng/mL)^{b} | Mean | 152.410 | 302.091 | 86.600 | 105.682 | 4.581 | 9.086 |
| Unadjusted | GM | 140.262 | 292.890 | 82.863 | 100.823 | 4.077 | 8.524 |
| Conc. | CV (%) | 45.47 | 24.43 | 34.16 | 31.05 | 55.49 | 34.88 |
| tₘₐₓ (h) | n | 10 | 11 | 10 | 11 | 10 | 11 |
| Baseline- | Median | 12.000 | 8.000 | 47.990 | 48.000 | 18.015 | 8.000 |
| adjusted Conc. | (min, max) | (4.0, 648.5) | (2.0, 24.1) | (24.0, 648.5) | (12.0, 48.0) | (0.98, 48.1) | (1.0, 24.1) |
| tₘₐₓ (h) | n | 10 | 11 | 10 | 11 | 10 | 11 |
| Unadjusted | Median | 12.000 | 8.000 | 47.990 | 48.000 | 18.015 | 8.000 |
| Conc. | (min, max) | (4.0, 648.5) | (2.0, 24.1) | (24.0, 648.5) | (12.0, 48.0) | (0.98, 48.1) | (1.0, 24.1) |
| AUC_{D 1-D 30} | n | 10 | 10 | 10 | 10 | 10 | 10 |
| (h*pg/mL or | Mean | 20793.725 | 37263.109 | 16942.583 | 22085.960 | 1142.290 | 1884.718 |
| h*ng/mL)^{b} | GM | 18425.279 | 36214.540 | 12634.405 | 18747.934 | 1119.875 | 1828.381 |
| Baseline- | CV (%) | 50.99 | 23.50 | 59.42 | 44.98 | 22.14 | 26.21 |
| adjusted Conc. | | | | | | | |
| AUC_{D 1-D 30} | n | 10 | 10 | 10 | 10 | 10 | 10 |
| (h*pg/mL or | Mean | 26791.539 | 43207.217 | 37085.593 | 38112.973 | 1180.511 | 1960.217 |
| h*ng/mL)^{b} | GM | 25408.792 | 41955.441 | 34849.696 | 36285.402 | 1158.677 | 1900.867 |
| Unadjusted | CV (%) | 38.57 | 25.94 | 41.02 | 29.62 | 21.28 | 25.92 |
| Conc. | | | | | | | |
| Cₛₛ (pg/mL or | n | 10 | 10 | 10 | 10 | 10 | 10 |
| ng/mL)^{b} | Mean | 20.379 | 30.920 | 22.148 | 25.248 | 1.322 | 2.084 |
| Baseline- | GM | 14.441 | 29.716 | 21.589 | 20.444 | 1.309 | 2.031 |
| adjusted Conc. | CV (%) | 83.80 | 28.23 | 74.97 | 48.70 | 14.72 | 23.90 |
| Cₛₛ (pg/mL or | n | 10 | 10 | 10 | 10 | 10 | 10 |
| ng/mL)^{b} | Mean | 28.627 | 39.228 | 50.617 | 47.995 | 1.376 | 2.192 |
| Unadjusted | GM | 25.183 | 37.205 | 46.215 | 45.432 | 1.362 | 2.126 |
| Conc. | CV (%) | 63.94 | 38.37 | 50.55 | 31.29 | 15.26 | 25.94 |

^{a}E2 and E1 parameters reported in pg/mL; progesterone parameters reported in ng/mL
^{b}GM = geometric mean
^{c}CV = coefficient of variation

TABLE 4
Summary of Plasma PK Parameters for Oral Comparator Arm (Pharmacokinetic Evaluable Population)

| | | Oral Comparator Treatment Arm^{a} (N = 11) | | | | | |
|---|---|---|---|---|---|---|---|
| Parameter | | E2 | | E1 | | P4 | |
| (unit) | Statistic | Day 1 | Day 29 | Day 1 | Day 29 | Day 1 | Day 29 |
| Cₘₐₓ (pg/mL | n | 11 | 11 | 11 | 11 | 11 | 11 |
| or ng/mL)^{b} | Mean | 18.903 | 47.848 | 167.878 | 296.424 | 10.217 | 13.209 |
| Baseline- | GM | 17.913 | 45.686 | 158.405 | 281.615 | 4.269 | 5.557 |
| adjusted Conc. | CV (%) | 32.91 | 30.38 | 36.21 | 33.16 | 117.67 | 135.21 |
| Cₘₐₓ (pg/mL | n | 11 | 11 | 11 | 11 | 11 | 11 |
| or ng/mL)^{b} | Mean | 25.164 | 51.282 | 185.909 | 314.455 | 10.258 | 13.250 |
| Unadjusted | GM | 22.877 | 49.216 | 177.438 | 300.105 | 4.385 | 5.668 |
| Conc. | CV (%) | 54.96 | 29.21 | 32.87 | 31.93 | 117.19 | 134.76 |
| tₘₐₓ (h) | n | 11 | 11 | 11 | 11 | 11 | 11 |
| Baseline- | Median | 10.000 | 6.000 | 6.000 | 4.000 | 2.000 | 1.020 |
| adjusted Conc. | (min, max) | (1.00, 24.0) | (0.50, 10.0) | (4.00, 10.0) | (2.00, 10.0) | (0.50, 10.0) | (0.50, 6.00) |
| tₘₐₓ (h) | n | 11 | 11 | 11 | 11 | 11 | 11 |
| Unadjusted | Median | 10.020 | 6.000 | 6.000 | 4.000 | 2.000 | 1.020 |
| Conc. | (min, max) | (4.00, 24.0) | (0.50, 10.0) | (4.00, 10.0) | (2.00, 10.0) | (0.50, 10.0) | (0.50, 6.00) |
| AUC_{0-24 H} | n | 11 | 11 | 11 | 11 | 10 | 11 |
| (h*pg/mL or | Mean | 366.453 | 849.479 | 2568.370 | 5012.178 | 17.245 | 19.010 |
| h*ng/mL)^{b} | GM | 347.969 | 810.760 | 2485.302 | 4774.390 | 10.995 | 12.031 |
| Baseline- | CV (%) | 34.53 | 31.50 | 26.16 | 32.36 | 96.01 | 90.76 |
| adjusted Conc. | | | | | | | |
| AUC_{0-24 H} | n | 11 | 11 | 11 | 11 | 10 | 11 |
| (h*pg/mL or | Mean | 454.291 | 932.404 | 3004.209 | 5449.140 | 18.387 | 20.041 |
| h*ng/mL)^{b} | GM | 436.720 | 895.385 | 2933.535 | 5224.620 | 12.897 | 13.778 |
| Unadjusted | CV (%) | 31.42 | 30.01 | 23.04 | 30.54 | 90.09 | 86.08 |
| Conc. | | | | | | | |
| C_{avg} (pg/mL or | n | NA | 11 | NA | 11 | NA | 11 |
| ng/mL)^{b} | Mean | NA | 35.4 | NA | 209 | NA | 0.792 |
| Baseline- | GM | NA | 33.8 | NA | 199 | NA | 0.501 |
| adjusted Conc. | CV (%) | NA | 31.5 | NA | 32.4 | NA | 90.8 |
| C_{avg} (pg/mL or | n | NA | 11 | NA | 11 | NA | 11 |
| ng/mL)^{b} | Mean | NA | 38.9 | NA | 227 | NA | 0.835 |
| Unadjusted | GM | NA | 37.3 | NA | 218 | NA | 0.574 |
| Conc. | CV (%) | NA | 30.0 | NA | 30.5 | NA | 86.1 |
| t_{1/2, eff} (h) | n | NA | 11 | NA | 11 | NA | 5 |
| Baseline- | Median | NA | 30.775 | NA | 22.672 | NA | 5.736 |
| adjusted Conc. | (min, max) | NA | (7.33, 47.0) | NA | (11.09, 34.27) | NA | (4.78, 176.59) |
| t_{1/2, eff} (h) | n | NA | 11 | NA | 11 | NA | 5 |
| Unadjusted | Median | NA | 26.139 | NA | 20.527 | NA | 5.166 |
| Conc. | (min, max) | NA | (4.27, 38.53) | NA | (10.5, 29.9) | NA | (3.76, 146.7) |

^{a}Oral Comparator: Oral Estrofem/Prometrium QD for 29 days (estradiol 1 mg tablet/progesterone 100 mg capsule).
^{b}E2 and E1 parameters reported in pg/mL; P4 parameters reported in ng/mL

The mean baseline-adjusted Css for E2 and E1 for 80/4 IVR was 20.4 and 22.1 pg/mL, respectively, and the corresponding values for 160/8 IVR were 30.9 and 25.2 pg/mL, respectively. For P4, the baseline-adjusted Css was 1.32 and 2.08 ng/ml for the 80/4 IVR and the 160/8 IVR, respectively. In comparison, mean trough baseline-adjusted concentrations of E2, E1, and P4 through Day 8 to Day 29 following once daily oral dosing of Estrofem/Prometrium (1 mg estradiol/100 mg progesterone) ranged from 26.8 to 58.6 pg/mL, 145 to 161 pg/mL, and 86 to 140 ng/ml, respectively. On Day 29, the mean baseline-adjusted Cavg was 35.4 pg/mL, 209 pg/mL, and 0.792 ng/ml for E2, E1, and P4, respectively, following once-daily oral dosing of Estrofem/Prometrium.

No deaths or SAEs were reported. Three participants (8.8%) were terminated early from the study: 1 (2.9%) due to an AE of upper respiratory tract infection not related to study drug after completing treatment with the 160/8 IVR, 1 (2.9%) due to withdrawal by subject after 15 days of treatment with the 160/8 IVR, and 1 (2.9%) prior to treatment due to meeting exclusion criterion for elevated ALT. Overall, 8/10 participants (80.0%) reported 30 TEAEs in the 80/4 IVR arm, 9/12 participants (75.0%) reported 47 TEAEs in the 160/8 IVR arm, and 8/11 participants (72.7%) in the oral reference arm reported 17 TEAEs. Notably, 1 participant in the 160/8 IVR accounted for 26 of the 47 total TEAEs in the 160/8 IVR treatment group. All TEAEs were mild or moderate in severity. Overall, the most frequently experienced TEAEs by SOC were reproductive system and breast disorders: 6/10 participants (60.0%) in the 80/4 IVR group experienced 14 TEAEs, 7/12 participants (58.3%) in the IVR Dose 2 group experienced 24 TEAEs, and 5/11 participants (45.5%) in the oral reference group experienced 6 TEAEs. Overall, the most frequently experienced TEAEs by PT was vaginal haemorrhage: 2/10 participants (20.0%) in the 80/4 IVR group experienced 2 TEAEs, 3/12 participants (25.0%) in the 160/8 IVR group experienced 3 TEAEs, and 1/11 participants (9.1%) in the oral reference group experienced 1 TEAE. One TEAE of intermenstrual bleeding occurred in the 160/8 IVR group.

### Discussion

Vaginal rings have been evaluated to treat either VMS or vaginal symptoms associated with menopause (i.e., VVA). The release rates in these studies varied considerably and depended on the indication. The treatment of VMS usually requires higher E2 release rates (from 50 to several hundred μg/d). Most IVRs evaluated and commercialized release E2 at rates ranging from about 50 to 150 μg/d. Most earlier studies of E2 administered via a number of routes of administration report concentrations in serum. In the present study, E2 (and E1 and P4) were measured in plasma. A comparison of serum and plasma E2 values has found that they are essentially equivalent. An IVR releasing the equivalent of E2 from 50 to 200 μg/d resulted in average serum concentrations of E2 in post-menopausal women of 38 to 126 pg/mL. This IVR is marketed as Femring® (Millicent Pharma) or Menoring® (Galen Ltd) depending on the country.

In one study examining the efficacy of rings that released estradiol-acetate equivalent to E2 at 50 or 100 μg/d in women aged <65 years experiencing ≥20 hot flushes/night sweats per week, it was found that the frequency of hot flushes/night sweats was significantly reduced (p<0.001) in both groups at 12 and 24 weeks from baseline. This corresponded to a reduction by 84% and 94% for the vaginal ring groups. The mean intensity of urogenital symptoms decreased from screening to the end of treatment in both groups. The incidence of adverse events was similar for both groups. No clinically relevant local effects of the vaginal ring were observed.

The 80/4 and 160/8 IVRs provided unadjusted E2 Css of 28.6 and 39.2 pg/mL, which is somewhat lower than observed from Femring/MenoRing although the 160/8 IVR gave similar E2 concentrations as the 50 μg/d Femring (38 pg/mL).

The results of the PK and safety assessments of two new IVRs in post-menopausal women provide evidence that plasma concentration of E2 over time should be sufficient to potentially treat women suffering from VMS as well as genitourinary symptoms of menopause. The data also suggest that release of P4 should provide adequate endometrial protection. The results of this study support the continued evaluation in women experiencing VMS.

### Example 2. Phase 1/2 Clinical Pharmacokinetic and Safety Study: 3 Months

The randomized, open-label, two-arm, parallel group Phase 1/2 study was designed to evaluate DARE-HRT1's safety, PK, and preliminary efficacy in improving the VMS as well as the vaginal symptoms of menopause in 21 healthy, post-menopausal women with intact uteri over approximately three consecutive months of use. The primary objective of the study was to describe the safety, tolerability, and PK of two different dose combinations (DARE-HRT1 IVR1 which delivers E2 80 μg/day with P4 4 mg/day versus DARE-HRT1 IVR2 which delivers E2 160 μg/day with P4 8 mg/day) over 12 weeks of use. Secondary objectives of the study were to assess the usability, participant tolerability, and preliminary effectiveness of DARE-HRT1 for both the VMS and vaginal symptoms of menopause. The study was registered at ClinicalTrials.gov (NCT05367973).

### Methods

Healthy postmenopausal women who did not use tobacco products and who were not taking exogenous hormones were enrolled. Menopause was defined by 12 months of spontaneous amenorrhea or 6 months of spontaneous amenorrhea with a plasma follicle stimulating hormone (FSH) concentration of 40 mIU/mL or higher. Participants had to have a body mass index of ≥18 and <38 kg/m², an endometrial thickness <4 mm on transvaginal ultrasound (TVUS), a normal mammogram within 2 years of the screening visit, and normal cervical cytology cancer screening. Baseline laboratories had to be either within normal limits or accepted by the investigator and medical monitor as not clinically significant. Participants with an endometrial thickness of 4-6 mm on TVUS at screening underwent an endometrial biopsy, which had to show benign histology (e.g. no hyperplasia, no polyps, no atypia, no carcinoma). Participants were excluded if they had any postmenopausal bleeding, a sexually transmitted infection (STI), a history of endometrial hyperplasia, and or significant cardiovascular, renal, pulmonary, neurological, or hepatic diseases preventing compliance with the study or impacting data quality. Eligible participants taking exogenous HT at study entry were required to undergo an appropriate washout period.

Participants used the study product for 12 weeks, across three 28-day cycles, with a new IVR administered on Day 1 of each cycle. For each cycle, Day 1 was defined as the first day of treatment, i.e., the day the IVR was self-administered by the participant. Participants were instructed to leave the IVR in place for 28 days and used a diary to document insertion and removal of the IVR and any instances of IVR expulsion, as well as any adverse events (AEs) or concomitant medication use.

In this open label, parallel-group, randomized study, participants who met study entry criteria were randomly assigned in a 1:1 ratio, to either DARE-HRT1 IVR1 (E2 80 μg/day with P4 4 mg/day) or DARE-HRT1 IVR2 (E2 160 μg/day with P4 8 mg/day). The randomization schedule was computer-generated using a permuted block algorithm. The study center was not a blocking factor in the randomization schedule. The randomization numbers were assigned sequentially by a member of the data management team who was not otherwise involved in the study.

The IVR component of DARE-HRT1 is an EVA copolymer ring. DARE-HRT1 IVRs were supplied as either IVR1 (E2 80 μg/day with P4 4 mg/day) or IVR2 (E2 160 μg/day with P4 8 mg/day by Sever Pharma Solutions (Malmö, Sweden). All study products were at refrigerated temperatures (2°−8° C.).

### Safety Assessments

Safety was assessed primarily through treatment emergent adverse events (TEAEs), which were graded as to their severity and relationship to product use. Any vaginal or vulvar irritation, vaginal discharge or pelvic pain was reported as an AE using the standardized Medical Dictionary for Regulatory Activities (MedDRA) codes and graded for severity. Adverse drug reactions (ADRs) were defined as all noxious and unintended responses to a medicinal product, for which a causal relationship between a medicinal product and an AE is at least a reasonable possibility, i.e., the relationship cannot be ruled out. All AEs judged by either the reporting investigator or the sponsor as having a reasonable causal relationship to an investigational product (IP) qualified as ADRs. An unexpected ADR was defined as an ADR for which the nature or severity is not consistent with the applicable product information (e.g., investigator brochure (IB) for an unapproved IP).

Safety was also measured by changes from screening in clinical laboratory assessments, vital signs, 12-lead electro-cardiogram (ECGs), physical examinations, and endometrial thickness width measurements by TVUS.

### Pharmacokinetic (PK) Evaluation

As illustrated in Table 5, participants were seen over three, 28-day cycles. PK analytes measured included estrone (E1), E2, and P4. During the first IVR use cycle, baseline PK sampling for E1, E2 and P4 occurred prior to a new IVR insertion on Day 1. These baseline values of E1, E2, and P4 were added to post dosing plasma concentrations of the analytes to adjust for each woman's endogenous hormonal state. On Day 1, plasma PK sampling was repeated at 0.5, 1, 2, 4, and 8 hours following IVR insertion. Participants also returned to the clinic 24 and 48 hours after a new IVR insertion for a single PK blood draw in each cycle. They also returned on days 8, 15 and 22 of each treatment cycle for a single PK blood draw and safety assessments, which included monitoring of AEs and concomitant medications, clinical laboratory findings, physical examinations, vital signs, speculum examinations, and TVUSs (and endometrial biopsies, if required).

TABLE 5
Schedule of Evaluations

| | Screening | Cycle 1 | | | Cycle 2 | | | | | Cycle 3 | | | EOT / ET | |
|---|---|---|---|---|---|---|---|---|---|---|---|---|---|---|
| | Study or Cycle Day | | | | | | | | | | | | | |
| | | | | 8, | | | | | | | | 8, | | |
| | Days −28 | | 2, | 15, | | 2, | | | | | 2, | 15, | | |
| Study Procedure | to −2 | 1 | 3 | 22 | 1 | 3 | 8 | 15 | 22 | 1 | 3 | 22 | 29 | 30 |
| Informed consent | X | | | | | | | | | | | | | |
| Medical and | X | X | | | | | | | | | | | | |
| surgical history | | | | | | | | | | | | | | |
| Eligibility | X | X | | | | | | | | | | | | |
| assessment | | | | | | | | | | | | | | |
| Physical | X | | | | | | | | | | | | X | |
| examination | | | | | | | | | | | | | | |
| Pap smear and | X | | | | | | | | | | | | | |
| STI screening | | | | | | | | | | | | | | |
| Speculum | X | X | | | X | | | | | X | | | X | |
| examination | | | | | | | | | | | | | | |
| Vaginal pH, | X | X | | | X | | | | | X | | | X | |
| vaginal cytology | | | | | | | | | | | | | | |
| Transvaginal | X | | | | | | | X | | | | | X | |
| ultrasound | | | | | | | | | | | | | | |
| 12-lead | X | | | | | | | | | | | | | |
| Electrocardiogram | | | | | | | | | | | | | | |
| Screening and | X | X | | X | X | | X | X | X | X | | X | X | |
| follow up plasma | | | | | | | | | | | | | | |
| laboratories: | | | | | | | | | | | | | | |
| Hematology and | | | | | | | | | | | | | | |
| blood chemistry | | | | | | | | | | | | | | |
| Randomization | | X | | | | | | | | | | | | |
| Dispense and | | X | | | X | | | | | X | | | | |
| Insert new IVR | | | | | | | | | | | | | | |
| (after pre-dose PK | | | | | | | | | | | | | | |
| sample) | | | | | | | | | | | | | | |
| Remove used | | | | | X | | | | | X | | | X | |
| vaginal | | | | | | | | | | | | | | |
| ring/return IVR(s) | | | | | | | | | | | | | | |
| PK blood samples | X | X | X | X | X | X | X | X | X | X | X | X | X | X |

EOT = End of Treatment;
ET = Early Termination

In cycle 3, day 29, which was the end of the 12-week study treatment (EOT), participants underwent safety assessments. PK blood sampling was done prior to removal of the last IVR and then was repeated at 0.5, 1, 2, 4, 8 and 24 hours after removal.

Plasma concentrations of E1, E2, and P4 were analyzed by Agilex Biolabs (Thebarton, South Australia) using dual tandem liquid chromatography, mass spectrometry (LC/MS-MS). The lower limit of quantification (LLOQ) for each analyte was as follows: E1 5 pg/mL, E2 2 pg/mL, and P4 0.025 ng/mL. For PK characterization, concentrations of analytes for each IVR dose were calculated using non-compartmental analysis (NCA). Concentrations that were below the LLOQ (BLQ) were imputed as 0.5*LLOQ. The PK parameter estimates were completed using WinNonlin (Pharsight Corporation). Actual sampling time was used for all parameter estimation. Standard PK parameters assessed included measures of the extent of absorption using estimates of the area under the plasma concentration-time curve (AUC), the maximum observed drug concentration (Cmax), the time to reach maximum drug concentration (Tmax) and the steady state concentration (Css).

### Sample Size and Statistical Analysis

A total of 20 healthy, postmenopausal female participants (N=10 per arm) were planned for inclusion in this study. The sample size for this study was based on feasibility and the goal to accurately assess the safety and PK of the DARE-HRT1 IVRs over a 12-week period. As such, the data are primarily descriptive in nature. The defined analysis populations for the primary objectives included: (1) the safety population, made up of all enrolled participants who received active treatment, i.e., inserted an IVR (and thus were exposed to at least one of the IVRs); and (2) the PK population, which was all participants who received a full course of study treatment for their dispensed treatment and who had sufficient concentration data for determination of PK parameters (primary endpoint).

Duration of exposure was calculated as the number of treatment days between first IVR insertion and last IVR removal. Total exposure to E2 and P4 was calculated as the analyte dose*duration of exposure. Finally, treatment compliance was calculated as 100%×(Total Exposure E2 [ug]/Total Required E2 [ug]; where total required=Planned Dose [ug]×84.

Summary statistics were described for each dosing cohort. Continuous variables included number of participants, mean, standard deviation (SD), median, minimum, and maximum. For categorical variables, we present the number and percentage of participants in each category. The denominator for percentage was based on the number of participants appropriate for the purpose of analysis. The study was not powered to demonstrate differences in the proportion of participants in each dosing group reporting various TEAEs and therefore, comparison of categorical variables for the safety analysis was done by Fisher's exact test. P values of <0.05 were considered significant.

### Results

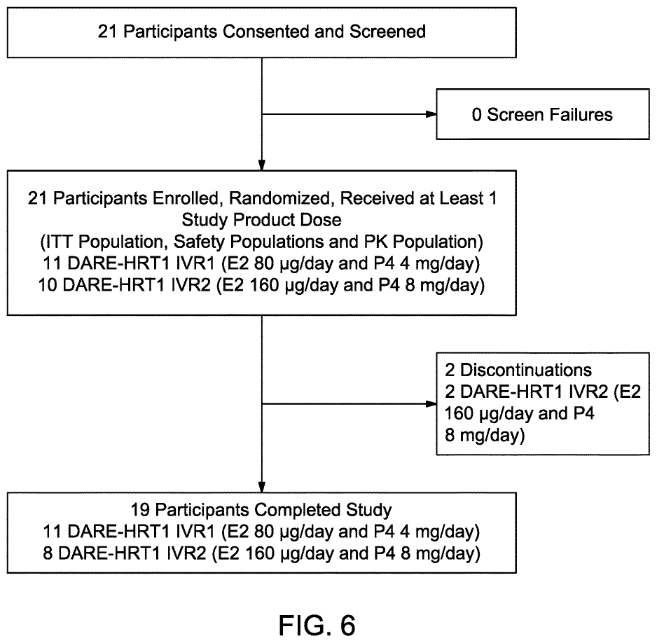

As shown in FIG. 6, 21 volunteers were screened and randomized to either DARE-HRT1-IVR1 (n=11) or DARE-HRT1-IVR2 (n=10). There were no screen failures. Table 6 displays the demographics of both dosing cohorts. All participants were menopausal based on 12 months of spontaneous amenorrhea.

TABLE 6
Summary of Demographic Data and Baseline
Characteristics (Randomized Population)

| | | | |
|---|---|---|---|
| | DARE-HRT1 IVR | DARE-HRT1 IVR | |
| | Dose 1 | Dose 2 | |
| | E2 80 μg/d + | E2 160 μg/d + | |
| | P4 4 mg/d | P4 8 mg/d | Overall |
| Parameter | (N = 11) | (N = 10) | (N = 21) |

Age (Years)

| | | | |
|---|---|---|---|
| Mean (SD) | 58.6 (3.41) | 59.0 (4.74) | 58.8 (4.00) |
| Median (Min, Max) | 59.0 (53, 64) | 59.5 (51, 65) | 59.0 (51, 65) |

Ethnicity (n [%])

| | | | |
|---|---|---|---|
| Not Hispanic or | 11 (100.0) | 10 (100.0) | 21 (100.0) |

Race (n [%])

| | | | |
|---|---|---|---|
| White | 11 (100.0) | 9 (90.0) | 20 (95.2) |
| Mixed Race | 0 (0.0) | 1 (10.0) | 1 (4.8) |

Weight (kg)

| | | | |
|---|---|---|---|
| Mean (SD) | 69.5 (10.4) | 76.7 (13.6) | 72.9 (12.3) |
| Median (Min, Max) | 68.5 (51.7, 85.9) | 73.6 (59.9, 99.6) | 72.8 (51.7, 99.6) |

Height (cm)

| | | | |
|---|---|---|---|
| Mean (SD) | 164 (4.02) | 163 (6.93) | 163 (5.47) |
| Median (Min, Max) | 164 (157, 172) | 161 (153, 175) | 163 (153, 175) |

Body Mass Index (kg/m²)

| | | | |
|---|---|---|---|
| Mean (SD) | 25.9 (3.53) | 28.9 (4.62) | 27.3 (4.26) |
| Median (Min, Max) | 25.0 (19.7, 30.8) | 27.2 (24.4, 37.5) | 26.5 (19.7, 37.5) |

### Safety Assessments

Table 7 demonstrates TEAEs, severity and relatedness to study product. Overall, 11/11 participants (100.0%) reported 35 TEAEs in the IVR1 group and 10/10 participants (100.0%) reported 62 TEAEs in the IVR2 group, with the proportion of participants in each dosing group reporting various categories of TEAEs being similar (Fisher exact p value=0.39). (Table 7). All TEAEs were graded as either mild or moderate. No serious AEs (SAEs) were reported.

TABLE 7
Frequency of Treatment Emergent Adverse Events in Both Dosing Groups

| Treatment | | |
|---|---|---|
| DARE-HRT1-IVR | DARE-HRT1-IVR | |
| Dose 1 | Dose 2 | |
| E2 80 μg/d + | E2 160 μg/d + | Fisher |
| P4 4 mg/d | P4 8 mg/d | Exact p |
| (N = 11) | (N = 10) | value |

Treatment Emergent Adverse Events (TEAEs)

| | N (%) | Number | N (%) | Number | Fisher |
|---|---|---|---|---|---|
| | Participants | of | Participants | of | Exact P |
| TEAE Description | Reporting | TEAEs | Reporting | TEAEs | Value |
| Any TEAE | 11 (100.0) | 35 | 10 (100.0) | 62 | 0.39 |
| Any Grade 3 or | 0 | 0 | 0 | 0 | |
| Higher TEAE | | | | | |
| Any Treatment-Related | 10 (90.9) | 20 | 9 (90.0) | 34 | |
| TEAEs (TEAEs with | | | | | |
| causality possibly | | | | | |
| related or related)^{a} | | | | | |
| TEAEs Leading to | 0 | 0 | 2 (20.0) | 4 | |
| Study Discontinuation | | | | | |
| TEAEs Leading to | 0 | 0 | 2 (20.0) | 3 | |
| Study Drug | | | | | |
| Discontinuation | | | | | |

^{a}= System Organ Class Preferred Term of these TEAEs listed in Table 8.

Two participants in the IVR2 dose group had TEAEs (continued breakthrough bleeding, nipple tenderness and depressed mood), considered related to study drug that led to study drug discontinuation and discontinuation from the study.

At baseline, the mean (SD) endometrial thickness width was 2.40 (1.26) and 2.11 (0.71) mm for IVR1 and IVR2, respectively. At the end of treatment, the mean (SD) endometrial thickness width was 3.03 (1.86) and 2.50 (0.98) for IVR1 and IVR2 respectively. All endometrial thickness measurements were ≤4.8 mm at the end of treatment.

Table 8 illustrates duration of treatment, calculated total exposure to E2 and P4, self-reported treatment adherence and the most common TEAEs by system/organ class (SOC). Some compliance numbers exceeded 100% because the EOT occurred after day 84 due to appointment scheduling logistics.

TABLE 8
Summary of Treatment Exposure, Compliance and System Organ
Class of Frequent Treatment Emergent Adverse Events

| Treatment | |
|---|---|
| DARE-HRT1-IVR | DARE-HRT1-IVR |
| Dose 1 | Dose 2 |
| E2 80 μg/d + | E2 160 μg/d + |
| P4 4 mg/d | P4 8 mg/d |
| (N = 11) | (N = 10) |

Duration of Exposure (days)

| | | |
|---|---|---|
| Mean (SD) | 85.1 (0.30) | 81.9 (9.33) |
| Median (Min, Max) | 85.0 (85, 86) | 85.0 (28, 85) |

Total Exposure of Estradiol (μg)

| | | |
|---|---|---|
| Mean (SD) | 6,807.3 (24.12) | 13,102.2 (1,493.33) |
| Median (Min, Max) | 6,800.0 (6,800, 6,880) | 13,600.0 (9,120, 13,600) |

Total Exposure of Progesterone (mg)

| | | |
|---|---|---|
| Mean (SD) | 340.4 (1.21) | 655.1 (74.67) |
| Median (Min, Max) | 340.0 (340, 344) | 680.0 (456, 680) |

Self-Reported Treatment Compliance

| | | |
|---|---|---|
| Mean (SD) | 101.30 (0.36) | 97.49 (11.11) |
| Median (Min, Max) | 101.19 (101.19, 102.38) | 101.19 (67.86, 101.19) |

Number of TEAEs per Dosing Group with causality of possibly
related or related to study product: System Organ Class Preferred Term

| | | |
|---|---|---|
| Reproductive system disorders | | |
| Overall Reproductive system | 12 | 23 |
| disorders | | |
| Vulvovaginal Discomfort or | 1 | 0 |
| Irritation | | |
| Vulvovaginal Pruritus | 2 | 0 |
| Nipple Soreness or Tenderness, | 2 | 10 |
| Mastalgia | | |
| Postmenopausal Bleeding | 2 | 9 |
| Pelvic Pain | 1 | 0 |
| Vulvovaginal Thrush | 2 | 0 |
| Vaginal Discharge | 2 | 4 |
| Nervous System Disorders | 5 | 8 |
| (e.g. Headache, Dizziness) | | |
| Psychiatric Disorders | 0 | 1 |
| (e.g Depressed Mood) | | |
| Vascular Disorders (e.g. | 0 | 1 |
| hot flush, hypertension) | | |
| Gastrointestinal Disorders | 3 | 0 |
| (e.g. Abdominal Cramping) | | |
| Musculoskeletal and Connective | 0 | 1 |
| Tissue Disorders (e.g. Low | | |
| Back Pain) | | |
| TOTAL | 20 | 34 |

### Pharmacokinetic Evaluations

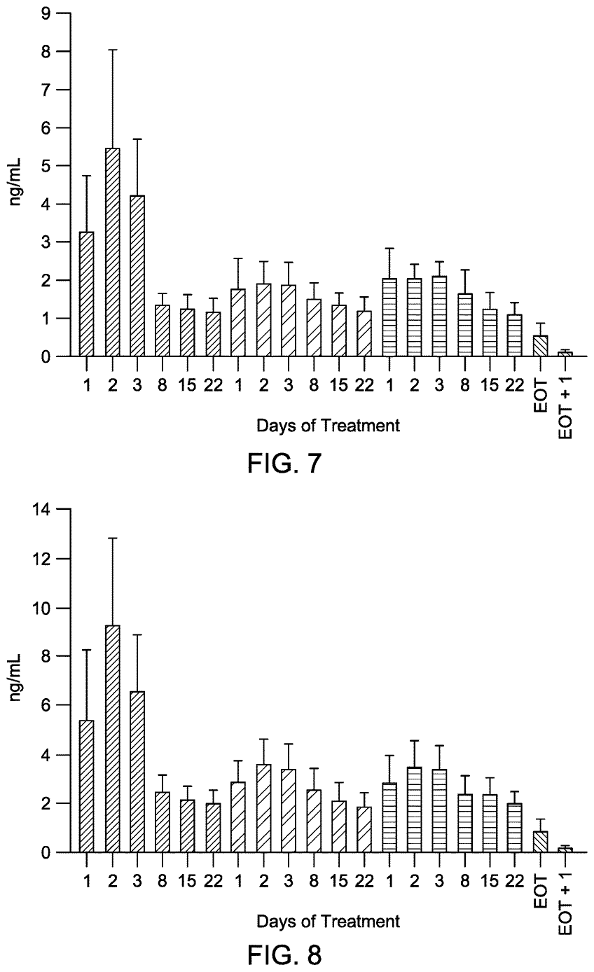

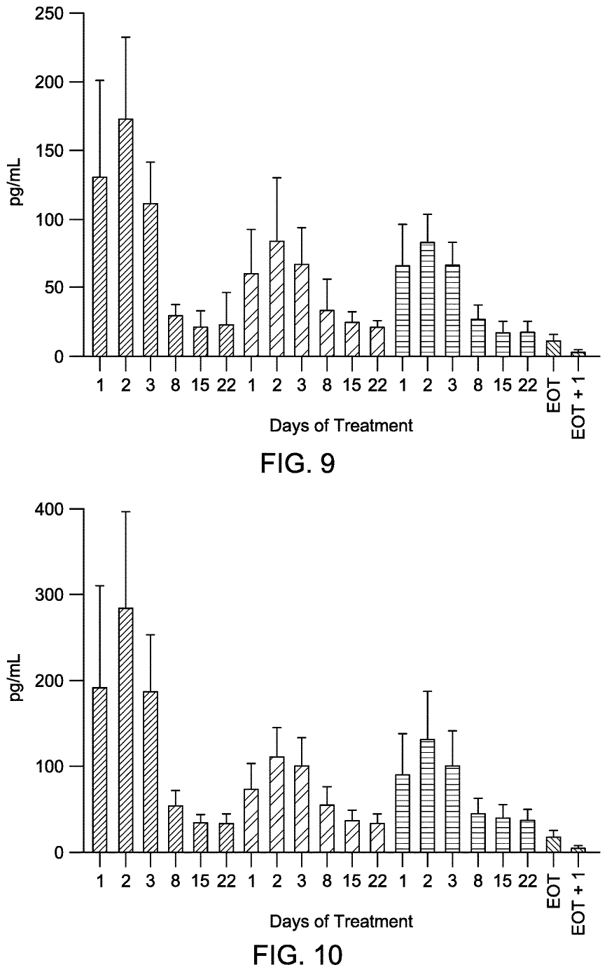

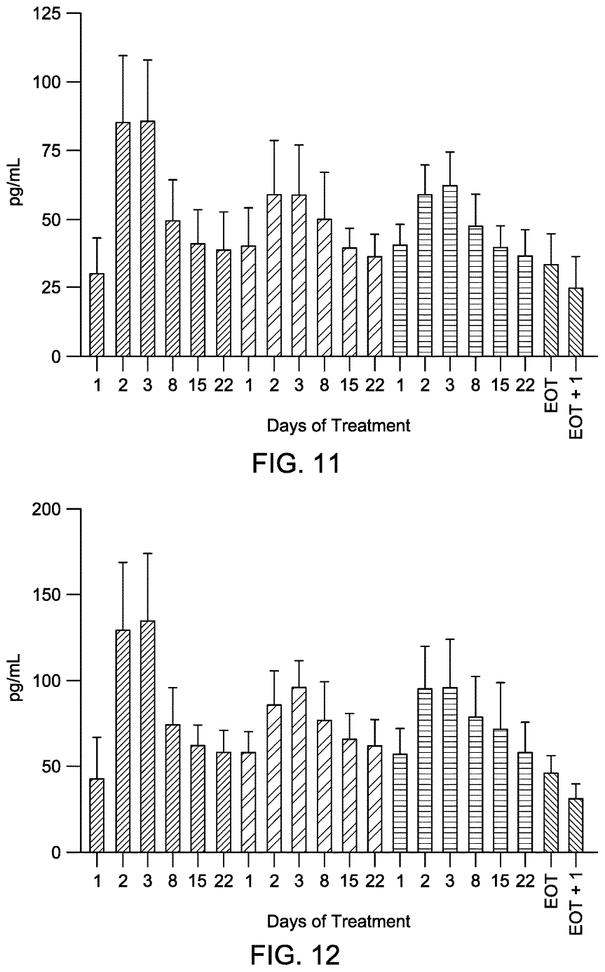

Tables 9, 10, and 11 and FIGS. 7-12 demonstrate the baseline adjusted plasma PK parameters and the mean (SD) baseline adjusted plasma concentrations versus time during each cycle of study product use for P4, E2, and E1. Table 9 and FIGS. 7 and 8 demonstrate that both IVRs resulted in baseline adjusted plasma P4 concentrations>1 ng/ml throughout the 12-week treatment. Table 10 and FIGS. 9 and 10 demonstrate that the baseline adjusted mean (SD) steady state (Css) plasma E2 concentrations achieved with the 160 μg/day E2 dose IVR was at least 37.35±8.96 pg/mL, with a range of 37.35-38.97 pg/mL over the 12-week treatment. The mean (SD) baseline adjusted, Css plasma E2 concentrations achieved with the lower 80 μg/day E2 dose IVR over each of the treatment cycles ranged from 22.17±4.47 pg/mL to 23.10±5.27 pg/mL).

TABLE 9
Summary of Baseline Adjusted Plasma Pharmacokinetic Parameters for Progesterone (ng/mL)

| | | Cycle 1 | | Cycle 2 | | Cycle 3 | |
|---|---|---|---|---|---|---|---|
| Parameter (unit) | Statistic | IVR Dose 1 | IVR Dose 2 | IVR Dose 1 | IVR Dose 2 | IVR Dose 1 | IVR Dose 2 |
| Progesterone | N (Unless | 11 | 10 | 11 | 9 | 10 | 8 |
| (ng/mL) | Specified) | | | | | | |
| Css (ng/mL) | N | 11 | 9 | 11 | 8 | 10 | 7 |
| | Mean | 1.10 | 1.89 | 1.12 | 1.70 | 1.25 | 1.80 |
| | SD | 0.28 | 0.49 | 0.31 | 0.23 | 0.34 | 0.28 |
| | Median | 1.10 | 1.88 | 1.14 | 1.72 | 1.19 | 1.89 |
| Cmax (ng/mL) | Mean | 5.57 | 8.95 | 2.56 | 3.50 | 2.83 | 3.40 |
| | SD | 2.44 | 3.38 | 0.94 | 0.55 | 0.87 | 0.34 |
| | Median | 4.62 | 8.01 | 2.52 | 3.30 | 2.81 | 3.51 |
| tmax (hours) | Median | 23.23 | 23.25 | 24.08 | 23.67 | 0.50 | 4.00 |
| Time to Cmax | Min, Max | 7.85, 24.2 | 23.00, 24.82 | 0.50, 48.30 | 1.00, 47.95 | 0.50, 23.03 | 0.48, 48.17 |
| AUCD 1-D 29 | Mean | 1,090 | 1,654 | 860 | 1,293 | 951 | 1,290 |
| (hours*ng/mL) | SD | 279 | 499 | 243 | 336 | 243 | 375 |
| Area Under the | Median | 1,164 | 1,585 | 888 | 1,309 | 878 | 1,491 |
| Curve between Day | | | | | | | |
| 1 and Day 29 | | | | | | | |

TABLE 10
Summary of Baseline Adjusted Plasma Pharmacokinetic Parameters for Estradiol (pg/mL)

| | | Cycle 1 | | Cycle 2 | | Cycle 3 | |
|---|---|---|---|---|---|---|---|
| Parameter (unit) | Statistic | IVR Dose 1 | IVR Dose 2 | IVR Dose 1 | IVR Dose 2 | IVR Dose 1 | IVR Dose 2 |
| Estradiol (pg/mL) | N | 11 | 10 | 11 | 9 | 10 | 8 |
| Css (pg/mL) | Mean | 23.00 | 38.11 | 23.10 | 37.35 | 22.17 | 38.97 |
| | SD | 8.49 | 8.45 | 5.273 | 8.964 | 4.468 | 10.79 |
| | Median | 22.28 | 35.28 | 23.93 | 36.26 | 20.73 | 38.16 |
| Cmax (pg/mL) | Mean | 70.90 | 113.98 | 43.59 | 70.10 | 45.33 | 75.37 |
| | SD | 19.35 | 38.95 | 15.66 | 22.194 | 9.52 | 25.99 |
| | Median | 74.00 | 113.70 | 40.80 | 64.90 | 42.95 | 77.27 |
| tₘₐₓ (hours) | Median | 24.20 | 47.09 | 48.48 | 47.95 | 47.26 | 47.26 |
| | Min, Max | 22.07, 48.05 | 23.07, 48.85 | 23.28, 167.08 | 23.23, 170.87 | 23.03, 48.43 | 23.07, 48.23 |
| AUCD 1-D 29 | Mean | 19,921 | 32,077 | 17,534 | 30,649 | 17,328 | 31,511 |
| (hours*pg/mL) | SD | 5,865 | 7,634 | 5,167 | 8,316 | 2,981 | 9,225 |
| | Median | 19,690 | 31,255 | 17,519 | 28,723 | 15,798 | 29,932 |

TABLE 11
Summary of Baseline Adjusted Plasma Pharmacokinetic Parameters for Estrone (pg/mL)

| | | Cycle 1 | | Cycle 2 | | Cycle 3 | |
|---|---|---|---|---|---|---|---|
| Parameter (unit) | Statistic | IVR Dose 1 | IVR Dose 2 | IVR Dose 1 | IVR Dose 2 | IVR Dose 1 | IVR Dose 2 |
| Estrone (E1) | N (Unless | 11 | 9 | 11 | 8 | 10 | 8 |
| pg/mL | Specified) | | | | | | |
| Css (pg/mL) | N | 11 | 9 | 11 | 8 | 10 | 7 |
| | Mean | 19.89 | 31.95 | 20.07 | 28.78 | 17.77 | 29.45 |
| | SD | 13.82 | 8.76 | 7.57 | 7.43 | 6.52 | 6.88 |
| | Median | 15.15 | 33.20 | 19.70 | 30.19 | 18.12 | 31.76 |
| Cmax (pg/mL) | Mean | 183.12 | 286.62 | 88.44 | 99.03 | 90.78 | 120.98 |
| | SD | 70.40 | 114.22 | 41.86 | 32.68 | 25.98 | 40.40 |
| | Median | 161.13 | 270.75 | 81.80 | 95.70 | 81.43 | 125.91 |
| tmax (hours) | Median | 7.92 | 15.57 | 23.23 | 23.73 | 15.52 | 15.46 |
| | Min, Max | 2.00, 23.75 | 4.00, 47.02 | 2.00, 24.10 | 7.93, 47.47 | 1.98, 24.82 | 2.00, 24.82 |
| AUCD 1-D 29 | Mean | 24,611 | 36,707 | 19,355 | 26,814 | 18,696 | 25,083 |
| (hours*pg/mL) | SD | 10,047 | 12,135 | 7,742 | 10,970 | 7,414 | 8,906 |
| | Median | 21,525 | 38,216 | 20,155 | 30,019 | 17,389 | 26,352 |

### Discussion

This 3-month study of two strengths of DARE-HRT1 demonstrated that both IVRs were safe over the treatment duration and released plasma E2 concentrations in the general range of the normal premenopausal follicular phase, approximately 20-80 pg/mL. Plasma P4 concentrations at steady state for both IVRs were in the range of the normal, post-ovulatory, luteal phase, >1 ng/mL, which supports that the in vivo release of P4 would protect the endometrium from the proliferative effects of the exogenous E2.

While all of the TEAEs were mild or moderate in severity, two participants in the IVR2 group elected to discontinue study product because they experienced known hormone related side effects. Product related TEAEs and severity of TEAEs were similar among the two dosing groups.

The levels of estradiol released from both the lower and higher dose formulation of DARE-HRT1 evaluated in the study achieved statistically significant improvement in VMS as well as the genitourinary symptoms of menopause, and vaginal pH and maturation index. Menopausal symptoms, including hot flashes and night sweats, were reduced compared with baseline in both DARE-HRT1 dose groups (p<0.01).

Participants also showed significant improvement from baseline in all measures surveyed on The Menopausal Quality of Life Survey (MENQOL), which surveys not only parameters of VMS, but also physical, psychosocial and sexual symptoms (p<0.01 on all domains). With DARE-HRT1 use, vaginal pH significantly decreased compared to baseline (p<0.01) and cytologic tests of the vaginal epithelium (vaginal maturation index) showed significant normalization (all p values<0.01 for increases in superficial cells, increases in intermediate cells and decreases in parabasal cells from baseline) among all participants. Finally, the most common genitourinary symptom, vaginal dryness, which was reported by 70% of participants at baseline, showed significant improvement in both DARE-HRT1 groups (p<0.01) and this subset also experienced significant decreases in vaginal pain with DARE-HRT1 use (p<0.01).

The study treatment was well tolerated with the types of most common adverse events consistent with other vaginal products. There were only two early discontinuations due to an adverse event, and no serious adverse events were reported.

DARE-HRT1 had a high level of acceptability in the study, with 100% of subjects reporting that the IVR was comfortable to wear, and there were no reports of the IVR being expelled from the vagina during use. Additionally, over 95% of subjects stated they would be either somewhat or very likely to use the IVR for a women's health condition or unrelated disease if needed.

Data from the study demonstrate that DARE-HRT1 successfully delivered estradiol and the progesterone over the 12 week evaluation period. The baseline-corrected steady state release of estradiol and progesterone from both the lower (IVR1) and higher (IVR2) dose versions of DARE-HRT1 evaluated in the study demonstrated steady state release levels in month 3 of the 12 week study as shown in Table 12 below:

TABLE 12
Summary of Month 3 Average Plasma Concentration

| | C_{avg} | |
|---|---|---|
| | (Steady State) | |
| DARE-HRT1 IVR1 (n = 11) | | |
| Estradiol | 22.17 (4.47) | pg/mL |
| Progesterone | 1.25 (0.34) | ng/mL |
| DARE-HRT1 IVR2 (n = 10) | | |
| Estradiol | 38.97 (10.79) | pg/mL |
| Progesterone | 1.80 (0.28) | ng/mL |

The levels of estradiol released from both the lower and higher dose formulation of DARE-HRT1 evaluated in the study achieved or exceeded the levels that were targeted for hormone therapy. Target levels of estradiol for hormone treatment for either the VMS or vaginal symptoms of menopause were established by reviewing PK levels published for FDA-approved products for both the treatment of VMS as well as the genitourinary symptoms of menopause. Based on the estradiol PK data in the DARE-HRT1 Phase 1/2 study, the results support the potential of DARE-HRT1 as an effective hormone therapy for both VMS and vaginal symptoms associated with menopause. The levels of progesterone released from both versions of DARE-HRT1 evaluated in the study met the objectives of releasing progesterone. Progesterone is used in hormone therapy to reduce the impact of estrogen on nontarget sites, such as the endometrium, to prevent estrogen-induced endometrial hyperplasia.

To effectively treat VMS in postmenopausal women, who typically have serum E2 concentrations of <20 pg/mL, exogenous E2 regimens must increase serum E2 concentrations into the premenopausal range. In healthy premenopausal women, serum E2 concentrations are normally 20-80 pg/mL in the early follicular phase of the menstrual cycle and increase to 150-500 pg/mL at ovulation. Thus, the DARE-HRT1-002 PK data demonstrate that the 160 μg/day E2 IVR put all participants in the normal, pre-menopausal, early follicular phase range for E2 (approximately 20-80 pg/mL), a level which is optimal to effectively treat VMS in menopause. On the contrary, the mean (SD) steady state plasma E2 concentrations resulting from use of the 80 μg/day E2 IVR left some IVR1 users in the menopausal range for plasma E2 (<20 pg/mL).

We measured the highest systemic absorption of E2 and P4 in the first two days of the first month of DARE-HRT1 IVR use. This is likely due to the fact that the study was in postmenopausal women, with a median age of 59 years. Although the participants did not have to meet FDA-defined criteria for VVA, baseline vaginal maturation index (VMI) parameters demonstrated few superficial cells and many parabasal cells, consistent with VVA. We have previously reported that menopausal women absorb vaginally applied pharmaceuticals more efficiently and rapidly than premenopausal women and after one month of vaginal estrogen use, vaginal absorption of topically applied drug is reduced, and is similar to that of premenopausal women. (Thurman, et al. J Acquir Immune Defic Syndr. 2018; 78 (1): 82-92.) Although we demonstrated higher absorption in the first few days of each cycle, this was most pronounced in cycle 1.

In terms of systemic safety, the DARE-HRT1-002 PK data also support that both the 160 μg/day and the 80 μg/day E2 IVRs are low dose products. Systemic safety of an exogenous E2 product is improved by the use of low dose formulations, which result in low, normal, premenopausal, early follicular phase E2 plasma concentrations (20-80 pg/mL E2), (e.g. conjugated equine estrogens (CEE) 0.3 mg or 0.45 mg daily), rather than higher dose exogenous E2 products (e.g. CEE 2.5 mg per day), which result in plasma E2 concentrations>150 pg/mL, consistent with ovulation. E2 related systemic severe AEs (e.g., pulmonary embolus, venous thromboembolism) and side effects (e.g., nausea and mastalgia) are directly related to steady state plasma E2 concentrations and participant age.

The data also support that both DARE-HRT1 IVRs deliver P4 concentrations at a level expected to protect the endometrium from endometrial hyperplasia. Specifically, in menopause and with anovulation (e.g. polycystic ovarian syndrome), serum P4 concentrations are <1 ng/ml (Verdonk, Vesper et al. 2019). In the normal, post ovulatory luteal phase of the menstrual cycle, serum P4 concentrations rise above 1 ng/mL, causing secretory, progesterone dominant and atrophic changes in the endometrium (Noyes, Hertig et al. 1975). Unopposed exogenous estrogen use and chronic anovulation (e.g. PCOS) are highly associated with endometrial hyperplasia because of the absence of P4 in these clinical situations. The PK data (Table 12) demonstrate that the mean (SD) steady state serum P4 concentrations achieved during either DARE-HRT1 IVR1 or DARE-HRT1 IVR2 use during the 12-week treatment period were all >1 ng/mL. These PK data demonstrate that both rings deliver sufficient P4 to counteract the proliferative effects of E2 to protect the endometrium from hyperplasia.

Endometrial biopsies were not performed in the present study, but all TVUS endometrial thickness measurements were ≤4.8 mm at screening and end of treatment. The fact that this IVR is a combination E2 and P4 product and that steady state plasma P4 concentrations were in the normal, post ovulatory range (>1 ng/mL) support that the dose of P4 will offset any proliferative impacts of the E2 on the endometrium.

The strengths of this study were that we achieved the primary objectives of measuring safety and systemic PK of two strengths of DARE-HRT1 in a healthy postmenopausal population. Although the sample size was small, consistent with early PK and safety studies, we obtained systemic PK data which was consistent with other recently approved oral and IVR VMS treatments. Participants used the study products over a 12-week study period, which is the normal period required to measure VMS treatment efficacy in placebo controlled studies. DARE-HRT1 is the first vaginal combination IVR of E2 and P4 hormones for the treatment of VMS in healthy postmenopausal women with an intact uterus. We believe this long-acting, non-oral VMS treatment regimen will fill an important gap in the options for treatment of this common menopause related condition.

### Example 3. Evaluation of the Preliminary Efficacy and Usability of DARE-HRT1

Preliminary efficacy was evaluated in the study described in Example 2, above.

Table 13 demonstrates significant improvement in vaginal cytology, as measured by the VMI and proportion of three vaginal cell types with use of either DARE-HRT IVR (all P values<0.01). Similarly, vaginal pH significantly decreased with DARE-HRT1 IVR treatment at both doses (all P values<0.01).

At baseline, 14 of 21 participants indicated that vaginal dryness was their MBS, two indicated that vaginal and/or vulvar itching was their MBS, and three indicated that vaginal pain associated with sexual activity (dyspareunia) was their MBS. Specifically, at baseline, IVR1 participants indicated that vaginal dryness (n=8/11), vaginal and/or vulvar itching (n=1/11), and vaginal pain associated with sexual activity (dyspareunia) (n=2/11) were their MBS. At baseline, IVR2 randomized participants reported that their MBS were as follows: vaginal dryness (n=6/10), vaginal and/or vulvar itching (n=1/10), and dyspareunia (n=1/10). Two participants in the IVR1 cohort reported vaginal bleeding with sexual activity at baseline, whereas no participants had this symptom at the end of treatment for either IVR group.

Among the 14 participants who reported that vaginal dryness was their MBS at baseline (eight from IVR1 cohort and six from IVR2 cohort), their median (interquartile range [IQR]) severity score was 2.0 (1, 3) at baseline and significantly decreased to a median (IQR) of 0 (0, 0) by the end of treatment with their respective IVR (P<0.01). These 14 women also experienced a significant decrease in dyspareunia from a median (IQR) severity of 1 (0, 2) at baseline to a median (IQR) severity of 0 (0, 0) at the end of treatment (P<0.01).

Table 13 demonstrates that when the self-reported subjective severity scores (among all participants) of all four GSM symptoms were compared as continuous variables between baseline and end of treatment, there was significant improvement in vaginal dryness and dyspareunia in the IVR1 dosing group (P values<0.01), with a nonsignificant trend in improvement (P=0.06) for the IVR2 dosing group. Unlike the changes in objective markers of vaginal pH and VMI (Table 1), there were no significant changes in self-reported severity of dysuria or vulvar or vaginal itching for either IVR group. IVR2 users experienced no significant changes from baseline in the severity of any of the four GSM symptoms (Table 13). This is likely due to the fact that only one woman in the IVR1 cohort reported mild dysuria at baseline, whereas no IVR2 participant reported the presence of this symptom at baseline. Similarly, although vulvar or vaginal itching was the MBS for two study participants (1 in each IVR group), the severity of vulvar or vaginal itching at baseline was reported as mild, moderate, or severe by two, one, and one IVR1 users respectively. For IVR2 users, one woman reported mild vulvar or vaginal itching, and one woman reported moderate vulvar or vaginal itching at baseline.

TABLE 13
Preliminary Markers of Local Estradiol Impact on Vaginal Epithelium

| | Baseline | | | End of treatment cycle | | | Difference (end | |
|---|---|---|---|---|---|---|---|---|
| | pretreatment | | | 3 (n = 8) or early | | | of treatment − | Wilcox [symbol] n |
| | cycle 1 day 1 (n = 11) | | | termination (n = 2) | | | cycle 1 day 1) | signed |
| IVR and Variable | Mean | SD | Median | Mean | SD | Median | Mean (95% CI) | rank sum P |

IVR1 (E₂ 80 μg/d with P₄ 4 mg/d)

| Vaginal cytology | | | | | | | | | |
|---|---|---|---|---|---|---|---|---|---|
| % Superficial cells | 0.20 | 0.42 | 0.0 | 14.3 | 11.68 | 15.0 | 15.3 | (7.2-23.4) | <0.01 |
| % Intermediate cells | 47.30 | 44.56 | 30.56 | 85.73 | 11.68 | 85.00 | 37.2 | (3.2-71.2) | <0.01 |
| % Parabasal cells | 52.40 | 44.54 | 69.50 | 0.00 | 0.00 | 0.00 | −52.4 | (−84.3 to −20.5) | <0.01 |
| Total VMI (%) | 23.85 | 22.33 | 15.25 | 57.14 | 5.84 | 57.50 | 34.42 | (25.7-40.3) | <0.01 |
| Vaginal pH | 5.40 | 0.63 | 5.30 | 4.67 | 0.00 | 4.70 | −0.73 | (−0.74 to −0.71) | <0.01 |

Self-reported severity of genitourinary symptoms (Likert score 0-3) by all participants

| | | | | | | | | | |
|---|---|---|---|---|---|---|---|---|---|
| Vaginal dryness severity | 1.9 | 1.04 | 2.0 | 0.1 | 0.30 | 0.0 | −2.3 | (−2.3 to −2.2) | <0.01 |
| Vaginal itching severity | 0.6 | 1.03 | 0.0 | 0.3 | 0.47 | 0.0 | −0.4 | (−0.4 to −0.4) | 0.63 |
| Dysuria severity | 0.1 | 0.30 | 0.0 | 0.0 | 0.0 | 0.0 | −0.1 | (−0.1 to −0.1) | >0.99 |
| Dysper [symbol] severity | 1.4 | 0.92 | 1.0 | 0.3 | 0.47 | 0.0 | −1.3 | (−1.3 to −1.3) | <0.01 |

IVR2 (E₂ 160 μg/d with P₄ 8 mg/d)

| Vaginal cytology | | | | | | | | | |
|---|---|---|---|---|---|---|---|---|---|
| % Superficial cells | 7.6 | 12.40 | 0.50 | 12.2 | 11.30 | 9.5 | 4.6 | (2.2-17.4) | <0.01 |
| % Intermediate cells | 42.05 | 37.92 | 50.75 | 87.20 | 11.33 | 89.50 | 45.2 | (20.8-69.5) | <0.01 |
| % Parabasal cells | 50.35 | 44.07 | 37.50 | 0.60 | 1. [symbol] | [symbol] . [symbol] 0 | −49.8 | (−80.8 to −18.7) | <0.01 |
| Total VMI (%) | 28.63 | 26.24 | 31.50 | 55.80 | 5.79 | 54.25 | 26.17 | (20.45 to 52.2) | <0.01 |
| Vaginal pH | 5.26 | 0.38 | 5.30 | 4.93 | 0.53 | 4.70 | −0.33 | (− [symbol] .32 to −0.32) | <0.01 |

Self-reported severity of genitourinary symptoms (Likert score 0-3) by all participants

| Vaginal dryness severity | 1.2 | 0.92 | 1.0 | 0.5 | 0.85 | 0.0 | −0.7 | (−0.7 to −0.7) | 0.06 |
|---|---|---|---|---|---|---|---|---|---|
| Vaginal itching severity | 0.3 | 0.67 | 0.0 | 0.2 | 0.63 | 0.0 | −0.2 | (−0.2 to −0.2) | >0.99 |
| Dysuria severity | 0.0 | 0.0 | 0.0 | 0.0 | 0.0 | 0.0 | NA | | NA |
| Dyspere [symbol] severity | 0.5 | 0.97 | 0.0 | 0.0 | 0.0 | 0.0 | −0.2 | (−0.2 to −0.2) | 0.25 |

IVR, intravaginal ring. [symbol]
indicates data missing or illegible when filed

### Preliminary Systemic (VMS) PD Estradiol Effects

The MENQOL questionnaire was used to assess preliminary efficacy of the study products for treatment of VMS. Table 14 demonstrates that there were significant decreases from baseline, indicating improvement in the overall MENQOL score and in all MENQOL domains for both DARE-HRT1 IVR. The largest improvement for both groups was in the MENQOL sexual domain, which consists of three questions regarding sexual desire, vaginal dryness, and avoiding intimacy due to physical symptoms.

Table 15 displays the frequency of responses to the VMS domain questions of the MENQOL questionnaire, questions 1-3. The most commonly reported VMS was night sweats (MENQOL question 2), and it was also the most severe symptom among participants reporting this symptom at baseline (Table 15). With the use of DARE-HRT1 IVR1, there was significant improvement in hot flashes (question 1) and night sweats (question 2) (P values 0.02 and 0.01 respectively), whereas there were no significant changes in sweating (question 3) (P=0.28) as 6 of 11 IVR1 users did not complain of this symptom at baseline (Table 15). Among DARE-HRT1-IVR2 users, there was significant improvement in the severity of hot flashes, night sweats, and sweating (all P values<0.03) (Table 15).

TABLE 14
Change from baseline to end of treatment in MENQOL domain scores based on dosing group

| | MENQOL score | | | MENQOL score (range, 0-6) | | | Difference (end | |
|---|---|---|---|---|---|---|---|---|
| | (range, 0-6) baseline | | | end of treatment (week 12) | | | of treatment − | Wilcox [symbol] n |
| | cycle 1 day 1 | | | or early termination | | | cycle 1 day 1) | signed |
| MENQOL DOMAIN | Mean | SD | Median | Mean | SD | Median | Mean (95% CI) | rank sum P |

DARE-HRT1-IVR1 (80 μg 17 [symbol] E₂/4 mg P₄) (n = 11 participants at baseline and week 12)

| | | | | | | | | | |
|---|---|---|---|---|---|---|---|---|---|
| Vasomotor symptom domain | 3.4 | 1.96 | 3.0 | 1.4 | 0.67 | 1.0 | −2.0 | (− [symbol] .2 to −2.8) | <0.01 |
| Psychosocial domain | 2.9 | 2.17 | 2.0 | 2.0 | 1.84 | 1.0 | −0.9 | (−1.2 to −0.6) | <0.01 |
| Physical domain | 3.5 | 1.69 | 3.0 | 1.8 | 0.75 | 2.0 | −1.6 | (−1.8 to −1.5) | <0.01 |
| Sexual domain | 4.5 | 2.02 | 5.0 | 1.3 | 0.47 | 1.0 | −3.02 | (−3.4 to −3.0) | <0.01 |
| Total MENQOL score | 3.5 | 1.29 | 3.0 | 1.6 | 0.67 | 2.0 | −1.8 | (−2.0 to −1.7) | <0.01 |

DARE-HRT1-IVR2 (160 μg 17 [symbol] E₂/4 mg P₄) IVR (n = 10 participants
at baseline, n = 8 participants at week 12, and n = 2 participants at early termination)

| | | | | | | | | | |
|---|---|---|---|---|---|---|---|---|---|
| Vasomotor symptom domain | 4.1 | 2.42 | 4.0 | 1.4 | 0.97 | 1.0 | −2.7 | (−3.0 to −2.4) | <0.01 |
| Psychosocial domain | 2.4 | 1.43 | 2.5 | 1.6 | 0.70 | 1.5 | −0.8 | (−1.0 to −0.6) | <0.01 |
| Physical domain | 3.0 | 1.49 | 2.5 | 2.2 | 1.40 | 2.0 | −0.8 | (−0.9 to −0.7) | <0.01 |
| Sexual domain | 4.3 | 2.16 | 4.0 | 1.7 | 1.25 | 1.0 | −2.6 | (−2.9 to −2.3) | <0.01 |
| Total MENQOL score | 3.5 | 1.43 | 3.0 | 1.8 | 0.92 | 2.0 | −1.7 | (−1.9 to −1.5) | <0.01 |

IVR, intravaginal ring; MENQOL, Menopause-Specific Quality of Life. [symbol]
indicates data missing or illegible when filed

TABLE 15
Frequency of responses to VMS domain of MENQOL at
baseline and end of treatment by dosing group

| | | Number of participants [symbol] | | |
|---|---|---|---|---|
| | | symptom was [symbol] | | Fisher's |
| [symbol] | [symbol] | [symbol] | [symbol] | exact P |

[symbol]

| | | | | |
|---|---|---|---|---|
| [symbol] | [symbol] | 5 | 10 | [symbol] |
| | 0 - Symptom [symbol] | 0 | 0 | |
| | 1 | 0 | 1 | |
| | 2 | [symbol] | 0 | |
| | 3 | [symbol] | 0 | |
| | 4 | 2 | 0 | |
| | 5 | 0 | 0 | |
| | 6 - Symptom [symbol] | 0 | 0 | |
| [symbol] | [symbol] | [symbol] | [symbol] | [symbol] |
| | 0 - Symptom [symbol] | 0 | 0 | |
| | 1 | 0 | 1 | |
| | 2 | 4 | 0 | |
| | 3 | 2 | 0 | |
| | 4 | 1 | 0 | |
| | 5 | 0 | 1 | |
| | 6 - Symptom [symbol] | 1 | 0 | |
| [symbol] | [symbol] | [symbol] | 10 | [symbol] |
| | 0 - Symptom [symbol] | 0 | 0 | |
| | 1 | 2 | 1 | |
| | 2 | 0 | 0 | |
| | 3 | 0 | 0 | |
| | 4 | 2 | 0 | |
| | 5 | 0 | 0 | |
| | 6 - Symptom [symbol] | 1 | 0 | |

[symbol]

| | | | | |
|---|---|---|---|---|
| [symbol] | [symbol] | [symbol] | 9 | [symbol] |
| | 0 - Symptom [symbol] | 0 | 0 | |
| | 1 | 2 | 0 | |
| | 2 | 0 | 0 | |
| | 3 | 2 | 1 | |
| | 4 | 1 | 0 | |
| | 5 | 0 | 0 | |
| | 6 - Symptom [symbol] | 2 | 0 | |
| [symbol] Night sweats | [symbol] not present | 2 | [symbol] | [symbol] |
| | 0 - Symptom [symbol] | 0 | 0 | |
| | 1 | [symbol] | 0 | |
| | 2 | [symbol] | 1 | |
| | 3 | [symbol] | 0 | |
| | 4 | [symbol] | 0 | |
| | 5 | [symbol] | 0 | |
| | 6 - Symptom [symbol] | 2 | 0 | |
| [symbol] | [symbol] | 3 | [symbol] | [symbol] |
| | 0 - Symptom [symbol] | 0 | 0 | |
| | 1 | 2 | 0 | |
| | 2 | 0 | 0 | |
| | 3 | 4 | 0 | |
| | 4 | 0 | 0 | |
| | 5 | 0 | 1 | |
| | 6 - Symptom [symbol] | 1 | [symbol] | |

MENQOL, [symbol] Quality of Life. [symbol]
indicates data missing or illegible when filed

### Usability and Acceptability Questionnaire

Table 16 demonstrates the frequency of responses, by DARE-HRT1 IVR dosing group, to the end of treatment (cycle 3, day 29) or early termination visit acceptability questionnaire. Most women agreed or strongly agreed that the study product was comfortable to wear, convenient to use, and worked with their lifestyle. Most women also reported that they were likely or very likely to use an IVR for treatment of both women's health and other health conditions. The acceptability responses were similar between the two dosing groups (all P values>0.22) at the end of treatment.

TABLE 16
Frequency of responses to usability and acceptability survey
by DARE-HRT1 dosing group at the end of treatment

| | | | Fisher's |
|---|---|---|---|
| [symbol] | [symbol] | [symbol] | exact P |
| [symbol] | | | |
| Strongly agree | 9 (81.8) | 7 (70.0) | 0.64 |
| Agree | 2 (18.2) | 3 (30.0) | |
| Neither agree nor disagree | 0 | 0 | |
| Disagree | 0 | 0 | |
| Strongly disagree | 0 | 0 | |
| [symbol] | | | |
| Strongly agree | 9 (81.8) | 6 (60.0) | 0.36 |
| Agree | 2 [symbol] | 4 (40.0) | |
| Neither agree nor disagree | 0 | 0 | |
| Disagree | 0 | 0 | |
| Strongly disagree | 0 | 0 | |
| [symbol] | | | |
| Strongly agree | 9 (81.8) | 6 (60.0) | 0.22 |
| Agree | [symbol] | 4 (40.0) | |
| Neither agree nor disagree | [symbol] | 0 | |
| Disagree | 0 | 0 | |
| Strongly disagree | 0 | 0 | |
| [symbol] | | | |
| Very comfortable | 8 [symbol] | [symbol] | 0.66 |
| Comfortable | 3 [symbol] | 4 (40.0) | |
| Neither comfortable or uncomfortable | 0 | 0 | |
| Uncomfortable | 0 | 0 | |
| Very uncomfortable | 0 | 0 | |
| [symbol] | | | |
| Very likely | 10 (90.9) | 7 (70.0) | 0.38 |
| Somewhat likely | 1 [symbol] | 2 (20.0) | |
| Neither likely nor unlikely | 0 | [symbol] | |
| Somewhat unlikely | 0 | 0 | |
| Very likely | 0 | 0 | |
| [symbol] | | | |
| Very likely | 9 [symbol] | 7 (70.0) | 0.78 |
| Somewhat likely | 2 [symbol] | 2 (20.0) | |
| Neither likely nor unlikely | 0 | 0 | |
| Somewhat unlikely | 0 | 1 (10.0) | |
| Very likely | 0 | 0 | |

[symbol]

indicates data missing or illegible when filed

### Discussion

This 3-month study of two strengths of DARE-HRT1 demonstrated that both IVR were acceptable and released enough E2 in vivo to demonstrate preliminary efficacy against both systemic (VMS) and local (GSM) menopausal symptoms.

We found that both the 80 μg/d and 160 μg/d E2 IVR resulted in significant decreases in vaginal pH and improvement of the VMI. We also found significant improvement in vaginal dryness and dyspareunia in the IVR1 dosing group and a non-significant trend in improvement for the IVR2 group. Thus, DARE-HRT1 might be an ideal product for healthy postmenopausal women with an intact uterus who have both symptomatic systemic (VMS) and local (GSM) hypoestrogenism. We recognize that participants did not have to have symptomatic GSM, as defined by a vaginal pH>5.0 and less than 5% superficial cells to enroll in this study, as is the normal criteria for estrogen GSM treatment studies, and therefore we interpret the GSM preliminary efficacy data with caution. The GSM data are, however, consistent with what is known about topical vaginal E2 treatment.

Similarly, participants did not have to have a benchmark frequency or severity of VMS, which is required in placebo-controlled VMS treatment efficacy studies. In fact, we recognize that not all women who were enrolled in this study had various VMS symptoms (eg, hot flushes, night sweats, sweating) present at baseline. However, among women who reported the presence of VMS symptoms at enrollment in the MENQOL questionnaire, the most severe and common systemic symptom was night sweats (MENQOL question 2), followed by hot flashes or flushes (MENQOL question 1), which are burdensome menopausal symptoms that result in overall poor health, due in part to chronic sleep deprivation and other symptom sequelae. Users of the 80 μg/d E2 IVR (DARE-HRT1-IVR1) did not experience significant improvement in their responses to the MENQOL question 3 (P=0.28), but this is likely due to the fact that this symptom was reported as not present by the majority (6/11) of these users at baseline.

Because VMS treatment efficacy was categorized as preliminary and was an exploratory end point in this study, we did not query participants on the frequency and severity of hot flashes at both 4 and 12 weeks of product use, as is required by the Food and Drug Administration (FDA) in placebo-controlled VMS treatment studies. This study was open-label, and the primary end points were to measure safety and PK. Because of the open-label, non-placebo-controlled design, interpretation of preliminary efficacy for VMS and GMS must be interpreted with caution. We used a Fisher's exact test to compare the frequency of the severity scores of the VMS domain of MENQOL, which is not impacted by sample size.

We used the MENQOL questionnaire to evaluate preliminary VMS treatment efficacy and note that the VMS MENQOL domain does not specify the frequency of VMS events. However, our data are encouraging that both IVR resulted in generalized improvement in all four of MENQOL domains. The MENQOL sexual domain showed the most improvement with treatment, and this domain has a question regarding vaginal dryness, which was the most bothersome genitourinary symptom reported by 14/21 participants at baseline.

DARE-HRT1 IVR were acceptable and tolerable for all participants with no significant differences in responses to the acceptability survey based on dosing group. This is not surprising as both IVR have the same physical characteristics.

The strengths of this study were that we achieved the objectives of measuring preliminary efficacy and acceptability of this first-in-category IVR in healthy postmenopausal women. We realize that participants did not have to meet the FDA-defined benchmarks for GSM or VMS symptoms, and therefore the preliminary efficacy data must be interpreted with caution. However, our preliminary efficacy data using surrogates of both systemic (VMS) and local (GSM) efficacy end points are consistent with what is known about both VMS and GSM treatment regimens. In addition, because preliminary efficacy and acceptability were exploratory end points, we did not power this study to detect difference between the two IVR in these variables. Participants used the study products over a 12-week study period, which is the normal period required by the FDA to measure both GSM and VMS treatment efficacy.

### CONCLUSIONS

The study product is the first combination E2 and P4 IVR product for the treatment of VMS symptoms. This nonoral VMS treatment regimen will likely fill an important gap in the options for treatment of this common menopause-related condition among postmenopausal women with an intact uterus. By delivering E2 and P4 by a nonoral route, this product meets recommended guidance from NAMS and other international organizations on the safe and effective treatment of VMS.

1. A method for treating one or more symptoms of menopause in a subject, comprising intravaginally inserting an implantable drug delivery device to the subject, wherein the implantable drug delivery device comprises an estrogen and a progestin, and further wherein the implantable drug delivery device provides a baseline-adjusted steady state plasma estrogen concentration between about 15 pg/mL and about 50 pg/mL in the subject.

2. The method of claim 1, wherein the implantable drug delivery device comprises a solid ethylene vinyl acetate polymer matrix.

3. The method of claim 1, wherein the subject is a female subject with a uterus.

4. The method of claim 1, wherein the one or more symptoms of menopause comprise vasomotor symptoms (VMS) and/or genitourinary syndrome.

5. The method of claim 1, wherein the one or more symptoms of menopause comprise hot flashes, night sweats, vaginal pH, changes in vaginal cytology, vaginal dryness, and vaginal pain.

6. The method of claim 1, wherein the estrogen is estradiol.

7. The method of claim 6, wherein the estradiol is bio-identical estradiol.

8. The method of claim 1, wherein the progestin is progesterone.

9. The method of claim 8, wherein the progesterone is bio-identical progesterone.

10. The method of claim 1, wherein the implantable drug delivery device delivers the estrogen and the progestin continuously over a period of time.

11. The method of claim 10, wherein the period of time is between 7 days and 35 days.

12. (canceled)

13. The method of claim 1, wherein the implantable drug delivery device comprises an ethylene-vinyl-acetate (EVA), intravaginal ring (IVR), wherein the ring contains at least two segments/fibers, wherein one segment contains the estrogen, and the second segment contains the progestin.

14. The method of claim 1, wherein the implantable drug delivery device comprises an inner core comprising one or more active pharmaceutical ingredients distributed throughout a first water-insoluble polymer; an intermediate coating positioned around the inner core, said intermediate coating comprising an acrylate polymer; and an outer coating positioned around the intermediate coating, said outer coating comprising a second water-insoluble polymer.

15. The method of claim 14, wherein the acrylate polymer is formed from one or more monomers of formula (I): 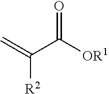
wherein R1 is selected from H, alkyl, alkenyl, alkynyl, or aryl; and R2 is selected from H or alkyl.

16. The method of claim 1, wherein the implantable drug delivery device releases the estrogen at a rate of about 50 μg/day to about 200 μg/day.

17. The method of claim 1, wherein the implantable drug delivery device releases the progestin at a rate of about 1 mg/day to about 15 mg/day.

18. (canceled)

19. (canceled)

20. (canceled)

21. (canceled)

22. The method of claim 1, wherein the implantable drug delivery device is replaced every 20 to 35 days.

23. (canceled)

24. The method of claim 1, wherein the implantable drug delivery device provides a baseline-adjusted steady state plasma estrogen concentration between about 20 pg/mL and about 30 pg/mL in the subject.

25. The method of claim 1, wherein the implantable drug delivery device provides a baseline-adjusted steady state plasma progesterone concentration between about 1 ng/ml and about 5 ng/ml in the subject.

26. (canceled)

27. The method of claim 1, wherein the one or more symptoms of menopause are improved by at least 20%.
